SEQUENCE LISTING (2 sequences)

SEQ ID NO: 1 - RNA, 19 nt, synthetic construct
  source 1..19: mol_type = other RNA
  modified_base 6: mod_base = OTHER; note = RNA (uracil)
gcaaatgcaaagaaggaaa

SEQ ID NO: 2 - RNA, 19 nt, synthetic construct
  source 1..19: mol_type = other RNA
  modified_base 4: mod_base = OTHER; note = RNA (uracil)
  modified_base 9..11: mod_base = OTHER; note = RNA (uracil)
  modified_base 17: mod_base = OTHER; note = RNA (uracil)
cagtcgcgtttgcgactgg

(19) United States
(12) Patent Application Publication
(10) Pub. No.: US 2024/0345084 A1
(43) Pub. Date: Oct. 17, 2024

(54) A METHOD OF DETECTING A POPULATION OF MACROPHAGES

(71) Applicants: Myriam Aouadi, Huddinge (SE); Agency for Science, Technology and Research, Singapore (SG)

(72) Inventors: Myriam Aouadi, Huddinge (SE); Camille Bleriot, Singapore (SG); Florent Ginhoux, Singapore (SG)

(21) Appl. No.: 18/683,027

(22) PCT Filed: Aug. 29, 2022
(86) PCT No.: PCT/SG2022/050619

§ 371 Date: Feb. 12, 2024

(30) Foreign Application Priority Data
Aug. 30, 2021 (SG) 10202109489Y

(51) Int. Cl. G01N 33/569

(52) U.S. Cl. CPC G01N 33/56972; G01N 2333/70596

(57) ABSTRACT

There is provided a method of detecting a population of macrophage in a sample comprising detecting and/or determining the expression of Cdh5 in the macrophage in the sample. Also disclosed is a kit for detecting and/or separating and/or depleting a population of a macrophage, a method of depleting a population of a macrophage, a method of improving the health of an obese and/or overweight subject, a method of determining the risk of obesity and/or a metabolic impairment related to obesity in a subject, and an animal model thereof.

### RELATED APPLICATIONS

This application is the U.S. National Stage of International Application No. PCT/SG2022/050619, filed Aug. 29, 2022, which designates the U.S., published in English, and claims priority under 35 U.S.C. § 119 or 365(c) to Singapore application Ser. No. 10/202,109489Y, filed Aug. 30, 2021. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 5975.1026-001 Corrected Sequence Listing.xml; created Mar. 6, 2024, 5,062 Bytes in size.

### TECHNICAL FIELD

The present disclosure relates broadly to a method of detecting a population of macrophages.

### BACKGROUND

Resident Tissue Macrophages (RTMs) are a diverse population of immune cells characterized by tissue-specific phenotypes and exhibiting a wide range of functions within their tissue of residence. However, tissues are complex environments and macrophage heterogeneity within the same organ has been overlooked so far.

The liver harbours a population of RTMs called Kupffer cells (KCs) lining liver sinusoids, specialized in detoxifying the blood traveling from the gut to the liver via the portal vein, which might contain microbionts, harmful entero-pathogens or toxic by-products of digestion. Thus, KCs can break down old or damaged red blood cells, engulf incoming threats and play a central role during inflammation notably in the development of liver pathologies such as viral hepatitis, fibrosis, hepatocellular carcinomas, alcohol-related disorders or non-alcoholic steatohepatitis (NASH)/fatty liver disease (NAFLD). However, even if the involvement of KC in these pathologies is undisputable, their precise modes of action remain largely unknown.

KCs derive from fetal liver monocytic precursors which acquire their identity early during embryonic development and maintain themselves throughout life. Although early post-natal circulating monocytes contribute a minor fraction of KCs shortly after birth, KC renewal is almost completely independent of bone-marrow derived cells in the steady state. However, under inflammatory conditions or when native embryonic KCs are depleted, monocyte-derived macrophages can replace dying embryonic KCs. Moreover, alongside KCs other minor populations of ontogenically and functionally unrelated macrophages including capsular macrophages, or even peritoneal macrophages recruited after injury, reside in the liver. This results in a mosaic of hepatic macrophage populations, heterogeneous in origin, phenotype and functions, among which KCs represent by far the most abundant one.

Within the murine KC population, the existence of distinct subsets at steady-state has been proposed in various studies but their conclusions have run into the difficulty to distinguish between embryonic KCs and monocyte-derived macrophages. Whether these distinct populations play different roles in the pathophysiology of liver diseases also remains elusive.

In human studies, while recent single cell transcriptomic studies suggest the existence of two major subsets of liver macrophages, the functions of these distinct subsets remain also elusive. Therefore, there is a need to provide a method of detecting a population of a macrophages. There is also a need to provide a method of detecting subpopulations of tissue or liver macrophages. In particular, there is a need to provide a method of detecting metabolically active macrophages.

### SUMMARY

In one aspect, there is provided a method of detecting a population of macrophage in a sample comprising detecting and/or determining the expression of Cdh5 in the macrophage in the sample.

In some examples, the method further comprises detecting and determining the expression of one or more markers comprising CD107a, CD107b, IGFBP7 (Insulin-like growth factor-binding protein 7), LYVE1, CD36, CD206 and/or ESAM in a macrophage in the sample.

In some examples, method further comprises detecting and determining a cell to be a macrophage in the sample by detecting the expression of a macrophage marker.

In some examples, the macrophage is a Kupffer cell (KC), optionally an embryonically derived Kupffer cell.

In some examples, the method further comprises detecting and determining a cell to be a macrophage in the sample by detecting the expression of Clec4f, Lyz2, Vsig4, Csf1r, Adgre1, F4/80, Tim4, Clec4F, and Vsig4.

In some examples, the method comprises detecting and determining a population expressing CD206lo and/or ESAM− to be a first population of a Kupffer cell and a population expressing CD206hi and/or ESAM+ to be a second population of a Kupffer cell.

In some examples, the method comprises detecting and determining a population expressing CD206lo and ESAM− to be a first population of a Kupffer cell and a population expressing CD206hi and ESAM+ to be a second population of a Kupffer cell.

In some examples, an over-expression of one or more markers comprising CD107a, CD107b, IGFBP7 (Insulin-like growth factor-binding protein 7), LYVE1, CD36, CD206 and/or ESAM determines a population to be a second population of a Kupffer cell.

In some examples, the method further comprises detecting, sorting, and/or determining the presence of one or more marker comprising CD206, ESAM, CD36, and combinations thereof.

In some examples, the method further comprises separating the first and/or the second population of macrophage, optionally wherein the method further comprises separating the first and/or the second population of a Kupffer cell.

In some examples, the method further comprises removing the population of cells expressing one or more of Cdh5+, CD107b+, CD206hi and/or ESAM+ from the sample.

In some examples, the method further comprises determining the expression of one or more markers comprising CD45, CD64, F4/80, TIM4, Clec4F, Adgre1 (F4/80), Timd4, Csf1r, and Clec4f, optionally the method further comprises removing and/or excluding cells that express one or more Adgre1+, Cx3cr1+, Timd4−, Clec4f−, and combination thereof.

In another aspect, there is provided a kit for detecting and/or separating and/or depleting a population of a macrophage, comprising providing an agent for detecting a population of a macrophage expressing Cdh5, optionally providing an agent capable of separating the population of the macrophage expressing Cdh5, and optionally providing an agent capable of depleting the population of the macrophage expressing Cdh5.

In some examples, the kit further provides an agent for detecting a population of a macrophage expressing CD107b+, CD206hi and ESAM+, optionally providing an agent capable of separating the population of the macrophage expressing CD107b+, CD206hi and ESAM+, and optionally providing an agent capable of depleting the population of the macrophage expressing CD107b+, CD206hi and ESAM+.

In yet another aspect, there is provided a transgenic animal model comprising a macrophage population expressing Cdh5 that have been genetically engineered to undergo ablation upon exogenous activation.

In yet another aspect, there is provided a method of depleting a population of a macrophage, comprising detecting and reducing a population of the macrophage in the subject, wherein the population of the macrophage expresses one or more Cdh5, CD107b, CD206, and ESAM.

In yet another aspect, there is provided a method of improving the health of an obese and/or overweight subject, comprising reducing a population of a macrophage in the subject, wherein the population of the macrophage expresses one or more Cdh5, CD107b, CD206, and ESAM.

In yet another aspect, there is provided a method of determining the risk of obesity and/or a metabolic impairment related to obesity in a subject, comprising detecting the expression level of Igfbp7/Cd36 expression in a macrophage.

In some examples, the method further comprises treating the subject identified to be of risk of obesity and/or the metabolic impairment related to obesity in the subject with an agent capable of depleting a macrophage cell expressing Cdh5.

In some examples, the method of any of the aspects disclosed herein reduces a CD206hi and ESAM+ macrophage, optionally the method reduces a Cdh5+, CD206hi, and ESAM+ Kupffer cell.

### DESCRIPTION OF EMBODIMENTS

In one aspect, there is provided a method of detecting a population of macrophage in a sample comprising detecting and/or determining the expression of Cdh5 in the macrophage in the sample.

As used herein, the term “expression” has been loosely used to refer to nucleic acid expression (such as gene expression and/or RNA expression) and protein expression.

In some examples, the method further comprises detecting and determining the expression of one or more marker comprising CD107a, CD107b, IGFBP7 (Insulin-like growth factor-binding protein 7), LYVE1, CD36, CD206 and/or ESAM in a macrophage in the sample. In some examples, the method comprises detecting and/or determining the expression of two or more, or three or more, or four or more, or five or more, or six or more, or all seven markers. In some examples, the method comprises detecting and/or determining the expression of 2, 3, 4, 5, 6, 7, or all markers as disclosed herein.

In some examples, the method further comprises detecting and determining a cell to be a macrophage in the sample by detecting the expression of a macrophage marker.

In some examples, the macrophage is a liver macrophage. Liver macrophages have long been considered as a homogeneous population of tissue scavengers in charge of the defence of liver against potential invaders coming from the portal vein downstream of the gut. Although this function is of importance, this is just one among a more and more detailed catalog of macrophage roles. There is now growing appreciation of the macrophage's ability to mediate tissue-specific homeostatic functions.

In some examples, the macrophage is a Kupffer cell (KC), optionally an embryonically derived Kupffer cell.

As known in the art, Kupffer cells (KCs) represent a heterogeneous population of immune cells highly adapted to their tissue of residence, the liver. This organ is the metabolic cornerstone of the organism and so KCs have a prominent role in many metabolic processes.

In some examples, the population of macrophage may be a first population of Kupffer cell (i.e., KC1) and/or a second population of Kupffer cell (i.e., KC2). In some examples, KC1 and KC2 may also express typical (or canonical) macrophage gene expression, RNA expression, and/or protein expression.

In some examples, the macrophage is a metabolically active macrophage.

As used herein, the term “canonical markers” or “typical markers” or “universal markers” is to be used in conjunction with one another and are interchangeably used to refer to markers that are known in the art to be expressed in most, if not all, subtypes of macrophages.

In some examples, the macrophage genes may be Clec4f, Lyz2, Vsig4, Csf1r and Adgre1 (F4/80), and the like. In some examples, the typical (canonical) and/or universal macrophage marker comprises one or more markers including, but is not limited to, F4/80, Tim4, Clec4F, and Vsig4, and the like.

In some examples, the method further comprises detecting and determining a cell to be a macrophage in the sample by detecting the expression of Clec4f, Lyz2, Vsig4, Csf1r, Adgre1, F4/80, Tim4, Clec4F, and Vsig4.

In the specific context of the murine liver, KCs are known to be localized in the sinusoids and are generally described as a homogeneous population of cells expressing specific markers such as F4/80, CD64 and Tim4. But the inventors of the present disclosure have observed a heterogeneity of CD64+ F4/80+ Tim4+ KCs, with a subpopulation expressing notably ESAM, LYVE1, CD206 and CD36 that the inventors of the present disclosure have named KC2 in opposition to the ESAM-LYVE1-CD206-Cd36-KC1. The inventors of the present disclosure have confirmed that both populations were bona fide Kupffer cells by using several fate-mapping systems known to trace macrophages according to their origins.

KC1 and KC2 were labelled at the same level confirming their nature of macrophages. But the inventors of the present disclosure have also observed that KC2 express many markers classically assigned to endothelial cells such as Esam, Lyve1 or Pecam1. Therefore, the inventors of the present disclosure have decided to use another available fate-mapping system based on one of these endothelial-associated genes, Cdh5. In this system, KC2 are efficiently labeled but not KC1, offering a powerful tool to distinguish the two populations.

Therefore, in some examples, the method comprises detecting and determining a population expressing CD206lo and/or ESAM− to be a first population of a Kupffer cell (i.e., KC1) and a population expressing CD206hi and/or ESAM+ to be a second population of a Kupffer cell (i.e., KC2). In some examples, the method comprises detecting and determining a population expressing CD206lo and ESAM− to be a first population of a Kupffer cell (i.e., KC1) and a population expressing CD206hi and ESAM+ to be a second population of a Kupffer cell (i.e., KC2).

Among the genes specifically expressed in the KC2 population, there is notably the gene Insulin Like Growth Factor Binding Protein 7 (igfbp7), a gene that has been very recently described as a key gene allowing the control of hepatocyte metabolism by liver macrophages. It has been shown that silencing specifically this gene in liver macrophages abolishes the obesity symptoms in high fat diet-fed mice by reprogramming hepatocytes. But another remarkable gene in the KC2-specific ones was Cd36, a fatty acid transporter responsible of the import of these lipids within cells.

Thus, in some examples, the method further comprises detecting and determining the expression of one or more markers comprising CD31, CD63, CD81, CD107a, Lyve1, IGFBP7 (Insulin-like growth factor-binding protein 7), CD36, and combinations thereof, and optionally wherein an over-expression of one or more markers determines a population to be a second population of a Kupffer cell (i.e., KC2). In some examples, the method further comprises detecting and determining the expression of one or more markers comprising CD107a, CD107b, IGFBP7, LYVE1, CD36, CD206 and/or ESAM and combinations thereof, and wherein an over-expression of one or more markers determines a population to be a second population of a Kupffer cell (i.e. KC2). In some examples, the method comprises detecting and/or determining the expression of two or more, or three or more, or four or more, or five or more, or six or more, or all seven markers. In some examples, the method comprises detecting and/or determining the expression of 2, 3, 4, 5, 6, 7, or all markers as disclosed herein.

In some examples, the second population of a Kupffer cell (i.e., KC2) is morphologically free of fenestrae on its surface. In some examples, the second population of a Kupffer cell (i.e., KC2) expresses LSEC-associated genes. In some examples, the LSEC-associated gene may include, but is not limited to, one or more of Mrc1, Pecam1 (CD31), Esam, Kdr, Lyve1, and combinations thereof.

In some examples, the method further comprises detecting, sorting, and/or determining the presence of one or more marker comprising CD206, ESAM, CD36, and combinations thereof.

In some examples, the method further comprises separating the first and/or the second population of macrophage, optionally wherein the method further comprises separating the first and/or the second population of a Kupffer cell.

In some examples, the method further comprises removing the population of cells expressing one or more of Cdh5, CD107b, CD206hi and ESAM+ (i.e., second population of Kupffer cell) from the sample.

In some examples, the method further comprises determining the expression of one or more markers comprising CD45, CD64, F4/80, TIM4, Clec4F, Adgre1 (F4/80), Timd4, Csf1r, and Clec4f.

In some examples, the method further comprises excluding (by removal or gating) of capsular macrophage. In some examples, a capsular macrophage expresses one or more genes comprising Adgre1+, Cx3cr1+, Timd4−, Clec4f−, and the like.

In another aspect, there is provided a kit for detecting and/or separating and/or depleting a population of a macrophage, comprising providing an agent for detecting a population of a macrophage expressing Cdh5, optionally providing an agent capable of separating the population of the macrophage expressing Cdh5, and optionally providing an agent capable of depleting the population of the macrophage expressing Cdh5.

In some examples, the kit further provides an agent for detecting a population of a macrophage expressing CD107b, CD206hi and ESAM+, optionally providing an agent capable of separating the population of the macrophage expressing CD107b, CD206hi and ESAM+, and optionally providing an agent capable of depleting the population of the macrophage expressing CD107b, CD206hi and ESAM+. In some examples, the kit may also comprise an agent for detecting a population of macrophage expressing one or more markers comprising CD107a, CD107b, IGFBP7, LYVE1, CD36, CD206 and/or ESAM and combinations thereof. In some examples, the kit comprises an instruction that an over-expression of one or more markers determines a population to be a second population of a Kupffer cell (i.e. KC2). In some examples, the kit may also comprise an agent for detecting two or more, or three or more, or four or more, or five or more, or six or more, or seven or more, or all markers as disclosed herein. In some examples, the kit may also comprise an agent for detecting two, three, four, five, six, seven, eight, or all markers as disclosed herein.

In addition of the specific labelling, the targeting of KC2 allowed the inventors of the present disclosure to develop a depletion model (Cdh5^{creERT2}xRosa^{DTR} mice) in which KC2 can be efficiently ablated. Thus, in yet another aspect, there is provided a transgenic animal model comprising a macrophage population expressing Cdh5 that have been genetically engineered to undergo ablation upon exogenous activation.

For example, the animal model may be such as, but is not limited to, mice, or rat, non-human primates, and the like. In some examples, the animal facility is under Specific-Pathogen-Free conditions. In some examples, the method may comprise the use of animal models such as, but is not limited to the background of, C57/BL6, BALB/c, CD-1, SCID, A/J, Sprague Dawley, Wistar, Rhesus monkey, Japanese monkey, Olive baboon, Squirrel monkey, Capuchin monkey, and the like. In some examples, the method comprises the use of C57/BL6 background mice.

In some examples, the transgenic animal model is a transgenic mouse model. In some examples, the mouse model is Cdh5^{creERT2}xRosa^{DTR} mouse. In some examples, the animal model allows for an inducible and specific depletion of a macrophage population. In some examples, the animal model allows for inducible and specific depletion of a Kupffer cell population, optionally a second population of Kupffer cell (i.e., KC2). In some examples, the Cdh5^{creERT2}xRosa^{DTR} mice have been obtained by breeding the Cdh5^{creERT2} mice (MGI: 3848982) with the Rosa^{DTR} mice (MGI: 3772576). In these animals, tamoxifen treatment will induce the expression of the Diphteria toxin receptor only in Cdh5 expressing cells. In some examples, depletion of cells including KC2 in Cdh5^{creERT2}xRosa^{DTR} mouse is triggered by injecting Diphteria toxin (DT) into a previously tamoxifen-treated mouse.

In yet another aspect, there is provided a method of depleting a population of a macrophage, comprising detecting and reducing a population of the macrophage in the subject, wherein the population of the macrophage expresses one or more Cdh5, CD107b, CD206, and ESAM.

In some examples, the population of the macrophage to be depleted is a second Kupffer cell population (i.e., KC2). In some examples, depletion of the second Kupffer cell population provides improvement to metabolic impairments in obesity. In some examples, the depletion of the second Kupffer cell population provides improvement in oxidative stress, improved glucose tolerance and/or less pronounced steatosis.

Strikingly, KC2−depleted mice did not gain weight when fed with high fat diet. Furthermore, glucose tolerance was improved and liver steatosis prevented in these mice. Of note, a specific silencing of Cd36 in KC had a comparable effect, even if of lower amplitude. This showed that CD36hi KC2 regulate liver oxidative stress associated with obesity via CD36 expression.

Obesity is a worldwide disease associated to high morbidity and its eradication constitutes one of the most important health challenges of the next century. But the etiology of the disease remains still elusive and a better comprehension is necessary to design effective therapeutic strategies. Macrophage heterogeneity is well appreciated across tissues but the diversity within the same tissue is often overlooked. This study reveals the coexistence of two subpopulations of Kupffer cells in murine liver with a minor CD206hiCD36hi one (KC2) endowed with metabolic functions and involved in regulating liver oxidative stress associated with obesity. In particular, the inventors of the present disclosure have identified a subpopulation of KCs specifically involved in the development of obesity (KC2) and developed a system to specifically label and deplete them, paving the way to a new strategy to defeat obesity.

Therefore, in another aspect, there is provided a method of improving the health of an obese and/or overweight subject, comprising reducing a population of a macrophage in the subject, wherein the population of the macrophage expresses one or more Cdh5, CD107b, CD206, and ESAM.

The inventors have also targeted CD36 expression on KC2 and show that CD36 pathway on KC2 regulate liver oxidative stress associated with obesity. Thus, in yet another aspect, there is provided a method of determining the risk of obesity and/or a metabolic impairment related to obesity in a subject, comprising detecting the expression level of Igfbp7 and/or Cd36 expression in a macrophage.

In some examples, the method further comprises treating the subject identified to be of risk of obesity and/or the metabolic impairment related to obesity in the subject with an agent capable of depleting a macrophage cell expressing Cdh5.

In some examples, the method reduces a CD206hi and ESAM+ macrophage, optionally the method reduces a Cdh5+, CD206hi, and ESAM+ Kupffer cell. In some examples, the method reduces a subpopulation of macrophages in a statistically significant amount. In some examples, the subpopulation of macrophages may be a KC1 and/or a KC2. In some examples, the reduction in the population of Kupffer cell is sufficient in improving the condition of (such as metabolic impairment of) the subject.

In some examples, the method may further comprise collecting a biological sample. In some examples, the biological sample may comprise a solid sample or a liquid sample. In some examples, the biological sample comprises a solid sample (such as liver sample).

In some examples, the method comprises determining a biological content (such as total triglyceride (TG) content) using a kit (such as colorimetric kit). In some examples, the biological content (such as TG content) is normalized against a concentration (such as protein concentration) as determined by a kit (such as Pierce BCA protein assay kit).

In some examples, the method comprises measuring intracellular amounts of a compound (such as H₂O₂, or malondialdehyde) using a kit (such as Amplex™ Red Hydrogen Peroxide/Peroxidase Assay Kit, or Lipid Peroxidation (MDA) Assay Kit (Colorimetric/Fluorometric)). In some examples, the method comprises performing a liquid sample (such as plasma multi-analyte) profiling using an analyzer (such as clinical chemistry analyzer) with an indicated kit (such as a colorimetric kit).

In some examples, the method comprises preparing a solid sample (such as hepatocyte) for analysis (such as flow cytometry analysis), by isolating the cells with centrifugation. In some examples, the solid sample (such as hepatocyte) were digested by enzymes (such as collagenase, or DNAse I) to isolate cells (such as macrophages). In some examples, the enzyme digestion is performed for such as, but is not limited to, 5 mins, or 10 mins, or 15 mins, or 20 mins, or 25 mins, or 30 mins, or 35 mins, or 40 mins, and the like. In some examples, the enzyme digestion is performed at a temperature such as, but is not limited to, 34° C., 35° C., 36° C., 37° C., 38° C., 39° C., 40° C., and the like. In some examples, the enzyme digestion is performed for 30 mins at 37° C. by running through a needle (such as a 18G needle).

In some examples, the isolated cells (such as macrophage) were directly stained with antibodies for analysis (such as flow cytometry) after cell (such as red blood cell) lysis. Data were generated with instruments known in the art (such as LSRII or Imagestream Amnis) and analysed by a software known in the art (such as Flow Jo). Cells were sorted using instrument known in the art (such as FACS Aria II or III).

In some examples, the method comprises preparing the biological sample (such as cells) for analysis (such as flow cytometry), labelling, and data recording. In some examples, the biological sample (such as cells) is stained with antibodies (such as for cisplatin to determine cell viability). Analysis was performed using a kit (such as Cytofkit) and an algorithm (such as One-sense).

In some examples, the method comprises processing the samples for genomics analysis (such as 10× genomic analysis) using a platform (such as Chromium Single Cell 3′ platform). In some examples, the method comprises pooling and sequencing cells (such as CD45+Tomato−, or CD45+Tomato+ cells) on lanes (such as Novaseq lanes) by a commercial company (such as Novagene AIT).

In some examples, the method comprises preparing the samples for translation analysis (such as RiboTag analysis). In some examples, the method comprises sorting and resuspending cells in a buffer (such as lysis buffer) and a compound (such as cycloheximide). In some examples, the cell homogenates were centrifuged (such as for 10,000 g, 4° C. for 10 min) to remove cell debris. In some examples, the supernatants were transferred on ice and antibodies (such as anti-HA, or mouse monoclonal IgC1 antibody) were added to the supernatant. In some examples, the incubation may comprise, but is not limited to, 1 h, or 2 h, or 3 h, or 4 h, or 5 h, or 6 h, or 7 h, or 8 h, or 9 h, or 10 h, or 11 h, or 12 h, and the like, in the cold room. In some examples, the sample with supernatant and added antibodies is incubated for 4 h in the cold room (such as at 4° C.) with slow rotation.

In some examples, the method comprises equilibration of magnetic beads (such as Dynabeads Protein G) to the sample by washing with a buffer (such as homogenization buffer). In some examples, the magnetic beads (such as Dynabeads Protein G) were added to the sample after incubation (such as 4 h) of the sample with antibodies. In some examples, the sample is further incubated overnight in the cold room (such as at 4° C.). In some examples, the samples were washed one time, or two times, or three times with a buffer (such as high-salt buffer). In some examples, the samples were washed three times (such as 5 min per wash) with a buffer (such as high-salt buffer) in a cold room on a rotator.

In some examples, the method comprises removing the excess buffer from the magnetized beads, and purified nucleic acid (such as RNA) was used as a sample (such as single cell sample). In some examples, the method comprises generating nucleic acid libraries (such as cDNA libraries) using a sequencing method (such as single cell RNA sequencing method).

In some examples, the method comprises capturing cells in a single run with barcoded samples (such as 12 samples) pooled together for an experiment (such as Rhapsody experiment). In some examples, the method comprises processing the samples according to the nucleic acid targeted (such as mRNA) and preparing the sample tag library with a kit (such as Rhapsody targeted mRNA and Abseq amplification kit). In some examples, the samples are then subjected to a run (such as indexed paired-end sequencing run) of cycles (such as 2×151 cycles) on an instrument (such as Illumina HiSeq 4000 system) with a spike in of a quality control (such as 20% PhiX).

In some examples, the method comprises labelling cells (such as KC1, KC2 population) for imaging (such as confocal immunofluorescence) by injection of antibodies (such as F4/80 Alexa Fluor 488, CD206-APC) into an animal (such as wild type C57BL/6 mice) prior to sacrifice of the animal.

In some examples, the method comprises fixing the sample (such as liver lobe) with a fixative (such as overnight with paraformaldehyde) and then incubating with a sugar solution (such as 30% sucrose for 24 h ). In some examples, the method comprises embedding the fixed samples (such as liver lobe) into an embedding compound (such as optimal cutting temperature (OCT)) and cut into sections with an instrument, or in low melting point gel (such as 4% agarose) for instrument sections.

In some examples, the method comprises fixing the sample in a fixative (such as glutaraldehyde for 1 h at room temperature) and a compound (such as osmium tetroxide 1 h at room temperature). In some examples, the method comprises dehydrating the sample through a series of graded alcohol (such as 25% to 100% ethanol) and dried using a dryer (such as CPD 030 critical point dryer). In some examples, the method comprises coating the surface that the cells are grown with a metal (such as 5 nm gold) by coating (such as sputter coating) with an instrument (such as SCD005 high-vacuum sputter coater). In some examples, the method comprises examining the coated samples with a microscope (such as field emission JSM-6701F scanning electron microscope) at a voltage (such as acceleration voltage of 8 kV) using a detector (such as in-lens secondary electron detector).

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### EXPERIMENTAL SECTION

### Material & Methods

### Animal Models

### Mice

All mouse experiments and procedures were approved by the Institutional Animal Care and Use Committee of the Biological Resource Center (Agency for Science, Technology and Research, Singapore) in accordance with the guidelines of the Agri-Food and Veterinary Authority and the National Advisory Committee for Laboratory Animal Research of Singapore (ICUAC No. 181402).

The C57/BL6 mice were obtained from the Jackson Laboratory. All mice were bred and housed in the Biological Resource Centre animal facility under Specific Pathogen-Free conditions and are maintained on a C57BL/6 background. All mice used in the in vivo experiments were aged 7 to 12 weeks unless specified. Mice were given high fat diet (60% fat—#D12492 Research Diets Inc.) ad libitum or tamoxifen-enriched diet (Teklad TD.130855 EnVigo) when specified. For the glucose tolerance test, mice were fasted overnight and then received 2 g·kg−1 of glucose by oral gavage according to (Andrikopoulos et al., 2008). Glycaemia was then measured at indicated timepoints.

### Indirect Calorimetric Measurement (Metabolic Chamber)

For the metabolic cage study, mice were housed individually in metabolic chambers and maintained on a 12-hr dark-light cycle with lights from 6 am to 6 pm at 22° C. Oxygen consumption, CO2 emission, food consumption, movement and energy expenditure were measured using TSA metabolic chambers (TSA System, Germany) in an open-circuit indirect calorimetric system.

Glucan-Encapsulated siRNA Particles (GERPs) Administration

GERPs were prepared as previously described by other studies known in the art. Mice fed with HFD for 8 weeks were first randomized according to their body weight and glucose tolerance. Then mice were treated with a total dose of 2 mg GERPs loaded with siRNA against Cd36 (5′-GCAAAUGCAAAGAAGGAAA-3′) (SEQ ID NO: 1) or with negative control (Scr: 5′-CAGUCGCGUUUGCGACUGG-3′) (SEQ ID NO: 2) (Dharmacon) (80 μg), and Endoporter (0,1 mM). Mice received six doses of fluorescently labelled (FITC) GERPs by i.v injections over 15 days.

### Mouse Biochemical Parameters

Liver samples were collected and immediately flash frozen. From these, total triglyceride (TG) content was determined by a commercially available colorimetric kit (Roche; TG 12016648). TG concentration was normalized against protein concentration as determined by the Pierce BCA protein assay kit (Thermofisher;23227) following the manufacturer's instructions. Intracellular amount of H2O2 was measured using Amplex™ Red Hydrogen Peroxide/Peroxidase Assay Kit (Life Technologies; A22188). Malondialdehyde content was measured using a Lipid Peroxidation (MDA) Assay Kit (Colorimetric/Fluorometric) (Abcam; ab118970). Plasma multi-analyte profiling was performed using a clinical chemistry analyzer (Mindray BS-240 Pro, BioSentec) with the indicated colorimetric kits (all from Biosentec). All assays were performed following manufacturer's instructions.

### Cell Preparation

### Cell Isolation, Flow Cytometry and Sorting of Macrophages

Standard labelling procedures were used to prepare the cells for flow cytometry analysis. Hepatocytes were isolated by centrifugation using a density gradient as previously described by other studies known in the art. For macrophages, liver lobules were digested in collagenase/DNAse I (0.2 mg·ml−1 of collagenase, 5 units·ml−1 of DNAse I and 10% FBS in RPMI) for 30 minutes at 37° C. and dissociation was finalised by several passages through a needle 18G. No enrichment was performed to avoid any cell loss and isolated cells were directly stained for flow cytometry after red blood cell lysis. The antibodies used are listed in the key resources table. Data were acquired by LSRII (BD Bioscience) or Imagestream Amnis (Merck) and analysed by Flow Jo (Tree Star, Inc.). Cells were sorted using a FACS Aria II or III (BD Bioscience).

### Cytometry by Time of Flight (CyTOF)

Cells were prepared, labelled and data recorded as previously described by other studies known in the art. Briefly, cells were prepared as for conventional flow cytometry and stained with cisplatin to determine cell viability. Antibodies used are listed in Table 1. Antibodies used were commercially available and were functionally complexed with metals (more detail can be found at PMID: 32607886/Methods Mol Biol. 2020;2164:87-99./doi:10.1007/978-1-0716-0704-6_10., the content of which is incorporated herein by reference). Results were analysed using CytofKit and One-sense algorithms.

### Mass Spectrometry

Sorted cell populations were lysed in Urea lysis buffer (8M Urea/Tris-HCl 50 mM, pH 8), reduced in presence of TCEP 20 mM for 20 min at room temperature and further alkylated with 55 mM chloroacetamide. Following dilution with 100 mM triethylammonium bicarbonate (TEAB, pH8.5; Sigma-Aldrich #T7408) samples were digested with Lysyl endopeptidase (LysC, Wako #129-02541) and Trypsin (Promega, #V5117) in ratio (1:100) for 4 h and 18 h respectively.

Samples were further acidified with trifluoroacetic acid (TFA Sigma-Aldrich #T6508; 1% v/v) spun down 14,000 RPM for 10 min at room temperature and desalted using HLB 96 well plate (Waters, #WAT058951). Following crude step high pH reverse phase fractionation (4 fractions, Reposil-Pur Basic C18 10 μm, Dr Maisch Gmbh #r10.b9.0025), each fraction was separated on a 50 cm (id 75 μm) EASY-Spray RP-C18 LC column (Thermo Scientific) in a 75 min gradient of solvent A (0.1% Formic acid in water) and solvent B (99.9% acetonitrile, 0.1% Formic acid in water) on Easy LC 1000 (Thermo Fisher Scientific), coupled to Obritrap Fusion Lumos mass spectrometer (Thermo Fisher Scientific).

Peak lists were generated using MaxQuant software version 1.6.7.0. Spectra were searched against target-decoy Mouse Uniprot database with following fixed modifications: Carbamidomethyl (C) and variable modifications: Oxidated (M), Deamidated (NQ) Acetyl (Nterminal protein). Maximum 2 missed cleavages were allowed, mass tolerance: 4.5 ppm mass deviation (after recalibration) for OT-MS survey scan and 0.5 Da for IT-MS/MS ion fragments. FDR was set to 1%. Label free quantitation (LFQ) was performed.

### Transcriptomics

### Library Preparation

For bulk RNAseq experiments, between 20,000 and 50,000 cells were FACS-sorted and total RNA was extracted using Arcturus PicoPure® RNA Isolation kit (Arcturus® Thermo Fisher Scientific, Waltham, MA, USA) according to manufacturer's protocol. All Mouse RNAs were analysed on Agilent Bioanalyser (Agilent, Santa Clara, CA, USA) for quality assessment with RNA Integrity Number (RIN) range from 5.8 to 6.7 and median of RIN 6.4. cDNA libraries were prepared using 2 ng of total RNA and 1 ul of a 1:50,000 dilution of ERCC RNA Spike in Controls (Ambion® Thermo Fisher Scientific, Waltham, MA, USA) using the Smart-Seq v2 protocol with the following modifications: 1. Addition of 20 μM template switch oligo (TSO); 2. Use of 200 pg cDNA with ⅕ reaction of Illumina Nextera XT kit (Illumina, San Diego, CA, USA).

The length distribution of the cDNA libraries was monitored using a DNA High Sensitivity Reagent Kit on the Perkin Elmer Labchip (Perkin Elmer, Waltham, MA, USA). All samples were subjected to an indexed paired-end sequencing run of 2×151 cycles on an Illumina HiSeq 4000 system (Illumina) (25 samples/lane).

For Smart-seq2 single cell analysis, 288 single cells were sorted on a 96-well plate and cDNA libraries were generated using the Smart-seq v2 protocol with the following modifications: 1. 1 mg/ml BSA Lysis buffer (Ambion® Thermo Fisher Scientific, Waltham, MA, USA); 2. Use of 200 pg cDNA with ⅕ reaction of Illumina Nextera XT kit (Illumina, San Diego, CA, USA).

The length distribution of the cDNA libraries was monitored using a DNA High Sensitivity Reagent Kit on the Perkin Elmer Labchip (Perkin 41 Elmer, Waltham, MA, USA). All samples were subjected to an indexed paired-end sequencing run of 2×151 cycles on an Illumina HiSeq 4000 system (Illumina, San Diego, CA, USA) (298 samples/lane). After QC filtering, 169 cells were used for analysis.

For 10× analysis, samples were processed using the Chromium Single Cell 3′ (v3 Chemistry) platform (10× Genomics, Pleasanton, CA). Briefly, 100,000 cells CD45+ Tomato− including the KC and 100,000 cells CD45+ Tomato+ were pooled and sequenced on Novaseq lanes by Novagene AIT. After QC filtering, 78,944 cells were used for analysis.

For RiboTag analysis, samples were prepared as described by other studies known in the art. Briefly, 50,000 cells were sorted and resuspended on ice in 1 ml of lysis buffer (50 mM Tris, pH 7.4, 100 mM KCl, 12 mM MgCl2, 1% NP-40, 1 mM DTT, 1:100 protease inhibitor (Sigma Aldrich), 200 units/ml RNasin (Promega) and 0.1 mg/ml cycloheximide (Sigma Aldrich) in RNase free water). To remove cell debris, homogenate was transferred to an Eppendorf tube and was centrifuged at 10,000 g and 4° C. for 10 min. Supernatants were transferred to a fresh Eppendorf tube on ice and 5 μl (=125 μg) of anti-HA antibody (H9658, Sigma Aldrich) or 5 μl (=1 μg) of mouse monoclonal IgG1 antibody (Sigma, Cat #M5284) was added to the supernatant, followed by 4 h of incubation with slow rotation in a cold room at 4° C. Meanwhile, Dynabeads Protein G (Thermo Fisher Scientific), 100 μl per sample, were equilibrated to homogenization buffer by washing three times.

At the end of 4 h of incubation with antibodies, beads were added to each sample, followed by incubation overnight in cold room at 4° C. Samples were washed three times with high-salt buffer (50 mM Tris, 300 mM KCl, 12 mM MgCl2, 1% NP-40, 1 mM DTT, 1:200 protease inhibitor, 100 units/ml RNasin and 0.1 mg/ml cycloheximide in RNase free water), 5 min per wash in a cold room on a rotator. At the end of the washes, beads were magnetized, and excess buffer was removed. Purified RNA was then treated as the single cell samples, considering the low amount of RNA harvested. cDNA libraries were generated using the above single cell RNA sequencing method except the use of 300 pg cDNA for Illumina Nextera XT kit.

For the Rhapsody experiment, all the process was done by following manufacturer's (BD Biosciences) protocol. 16,775 cells were captured in a single run with 12 barcoded samples pooled together. The sample was processed according to BD mRNA targeted and sample tag library preparation with the BD Rhapsody™ targeted mRNA and Abseq amplification kit (Doc ID: 210969 Rev 3.0). Samples were then subjected to an indexed paired-end sequencing run of 2×151 cycles on an Illumina HiSeq 4000 system (Illumina, San Diego, CA, USA) with 20% PhiX spike in.

### Analysis of Transcriptomic Data

Raw reads were aligned to the mouse reference genome GRCm38_M13 from GENCODE using STAR 2.5.3a. Gene expression values in transcripts per million (TPM) were calculated using the same RSEM program Dimensionality reduction (PCA, tSNE and UMAP), clustering and differentially expressed genes (DEGs) analysis were conducted using Seurat version 2.4.3. Wilcoxon rank sum test was performed to calculate differentially expressed genes p-value. After got the p-value, p-value adjustment was performed using Bonferroni correction based on the total number of genes in the dataset and adjusted p-value <0.05 was used as a threshold for statistical significance. 10× and SMARTSeq2 data integration was conducted using Seurat version 3.0.1 with standard integration pipeline.

### Imaging

### Confocal Imaging

KC1 and KC2 were labelled for confocal immunofluorescence imaging by i.v. injection of 2 μg F4/80 Alexa flour 488 (Biolegend #123120) and 2 μg CD206-APC (Biolegend #141708) into WT C57BL/6 mice 10 min prior to sacrifice of the animal. Liver lobes were fixed overnight in PBS with 4% paraformaldehyde, then incubated for 24h in PBS with 30% sucrose. Subsequently liver lobes were either embedded into O.C.T (Killik Bio-Optica #05-9801) and cut into 60 μm thick sections with a cryostat at −14° C. or embedded in 4% low-melting-point agarose (Sigma-Aldrich) for 200 μM thick vibratome sections.

For O.C.T-embedded tissues, sections were blocked for 15 min with blocking buffer (PBS, 0.5% BSA, 0.3% Triton), then stained for 60 min at RT with CD38 Alexa flour 594 (Biolegend #102725) in wash/stain buffer (PBS, 0.2% BSA, 0.1% triton), washed twice for 5 min, stained with DAPI (Sigma #28718-90-3) for 5 min, washed again and mounted for imaging with Fluorosave™ Reagent (Millipore #345789). Images were acquired with an SP5 confocal microscope (Leica) with an 63× oil-immersion objective. For visualization purposes and to compensate for uneven slide illumination, layer fluorescent intensity was normalized using Imaris Normalize Layers tool. Subsequently autofluorescence was filtered from the image by channel 42 subtraction of a deep red autofluorescent channel from APC signal with Imaris Channel Arithmetics tool.

For agarose-embedded tissues, sections were permeabilized for 1 h in PBS supplemented with 0.4% Triton X-100 (Sigma-Aldrich) and 3% BSA (Sigma-Aldrich) and preincubated for 1 h in the blocking buffer (PBS supplemented with 3% BSA). Then tissues were labeled with the appropriate primary and secondary antibodies for 2 h at room temperature.

### Scanning Electron Microscopy

Cells were fixed in 2.5% glutaraldehyde in 0.1 M phosphate buffer for 1 hr (pH 7.4) at room temperature, treated with 1% osmium tetroxide (Ted Pella Inc) at room temperature for 1 hr, and then dehydrated through a graded ethanol series from 25% to 100% and dried using a CPD 030 critical point dryer (Bal-Tec AG, Liechtenstein). The surface on which the cells were grown, and the adhesive surface was coated with 5 nm of gold by sputter coating using a SCD005 high-vacuum sputter coater (Bal-Tec AG). The coated samples were examined with a field emission JSM-6701F scanning electron microscope (JEOL Ltd., United States) at an acceleration voltage of 8 kV using the in-lens secondary electron detector.

### Statistical Analysis

DEG analyses were performed using the Seurat v3 package. All DEGs obtained from tpm/count matrixes were calculated on normalised values with a logFC threshold of 0.25. Wilcoxon rank sum test was performed to calculate differentially expressed genes p-value. After obtaining the p-value, p-value adjustment was performed using Bonferroni correction based on the total number of genes in the dataset and adjusted p-value <0.05 was used as a threshold for statistical significance.

TABLE 1
(Key Resources Table)

| | | |
|---|---|---|
| REAGENT or | SOURCE | IDENTIFIER |

Antibodies

| | | |
|---|---|---|
| BUV395 Rat Anti-Mouse | BD | BD Biosciences Cat# 564279, |
| CD45 | Biosciences | RRID:AB_2651134 |
| BV650 anti-mouse/human | BioLegend | BioLegend Cat# 101239, |
| CD11b Antibody | | RRID:AB_11125575 |
| PE-CF594 Hamster Anti- | BD | BD Biosciences Cat# 562286, |
| Mouse CD3e | Biosciences | RRID:AB_11153307 |
| PE-CF594 Rat Anti- | BD | BD Biosciences Cat# 562291, |
| Mouse CD19 | Biosciences | RRID:AB_11154223 |
| PE-CF594 Rat Anti- | BD | BD Biosciences Cat# 562700, |
| Mouse Ly-6G | Biosciences | RRID:AB_2737730 |
| PE-CF594 Rat Anti- | BD | BD Biosciences Cat# 562453, |
| Mouse CD49b | Biosciences | RRID:AB_11153857 |
| APC/Cyanine7 anti- | BioLegend | BioLegend Cat# 123117, RRID:AB_893489 |
| mouse F4/80 Antibody | | |
| BV711 anti-mouse CD64 | BioLegend | BioLegend Cat# 139311, RRID:AB_2563846 |
| (FcyRI) Antibody | | |
| PE/Cyanine7 anti-mouse | BioLegend | BioLegend Cat# 130009, RRID:AB_2565718 |
| Tim-4 Antibody | | |
| AF700 anti-mouse I-A/I-E | BioLegend | BioLegend Cat# 107622, RRID:AB_493727 |
| Antibody | | |
| BV605 anti-mouse CD31 | BioLegend | BioLegend Cat# 102427, RRID:AB_2563982 |
| Antibody | | |
| PerCP/Cyanine5.5 anti- | BioLegend | BioLegend Cat# 128012, RRID:AB_1659241 |
| mouse Ly-6C Antibody | | |
| PE ESAM Monoclonal | Thermo | Thermo Fisher Scientific Cat# 12-5852-82, |
| Antibody | Fisher | RRID:AB_891537 |
| | Scientific | |
| AF647 anti-mouse CD206 | BioLegend | BioLegend Cat# 141712, |
| (MMR) Antibody | | RRID:AB_10900420 |
| AF488 LYVE1 Monoclonal | Thermo | Thermo Fisher Scientific Cat# 53-0443-82, |
| Antibody | Fisher | RRID:AB_1633415 |
| | Scientific | |
| PE/Cyanine7 anti-mouse | BioLegend | BioLegend Cat# 102615, RRID:AB_2566121 |
| CD36 Antibody | | |
| Anti-Mouse CD45 (30- | Fluidigm | Fluidigm Cat# 3089005B, RRID:AB_2651152 |
| F11)-89Y | | |
| CD19 Monoclonal | Thermo | Thermo Fisher Scientific Cat# Q10379, |
| Antibody (6D5) | Fisher | RRID:AB_10563403 |
| | Scientific | |
| Purified anti-mouse CD48 | BioLegend | BioLegend Cat# 103402, RRID:AB_313017 |
| InVivoMab anti-mouse | Bio X Cell | Bio X Cell Cat# BE0178, RRID:AB_10949066 |
| MHC class II (I-A) | | |
| Purified anti-mouse | BioLegend | BioLegend Cat# 108502, RRID:AB_313383 |
| CD107b (Mac-3) | | |
| InVivoMab anti-mouse IL- | Bio X Cell | Bio X Cell Cat# BE0051, RRID:AB_1107698 |
| 12 p40 | | |
| Purified anti-mouse Ly- | BioLegend | BioLegend Cat# 108101, RRID:AB_313338 |
| 6A/E | | |
| Purified anti-mouse CD88 | BioLegend | BioLegend Cat# 135802, RRID:AB_1953295 |
| Rat Anti-Ly-6G | BD | BD Biosciences Cat# 551459, |
| Monoclonal Antibody | Biosciences | RRID:AB_394206 |
| Purified anti-mouse Ly-6C | BioLegend | BioLegend Cat# 128002, RRID:AB_1134214 |
| CD9 antibody | BD | BD Biosciences Cat# 553758, |
| | Biosciences | RRID:AB_395032 |
| Siglec-F antibody | BD | BD Biosciences Cat# 552125, |
| | Biosciences | RRID:AB_394340 |
| LEAF ™ Purified anti- | BioLegend | BioLegend Cat# 130004, RRID:AB_1227800 |
| mouse Tim-4 | | |
| Purified anti-mouse | BioLegend | BioLegend Cat# 108902, RRID:AB_313409 |
| CD49b | | |
| CD11b antibody | BD | BD Biosciences Cat# 553308, |
| | Biosciences | RRID:AB_394772) |
| CD86 antibody | BD | BD Biosciences Cat# 553689, |
| | Biosciences | RRID:AB_394991 |
| Rat Anti-Mouse CD68 | Bio-Rad | Bio-Rad Cat# MCA1957, RRID:AB_322219 |
| Monoclonal antibody | | |
| BST2 Antibody | Novus | Novus Cat# DDX0390P-100, |
| (120G8.04) | | RRID:AB_2827525 |
| Mouse Mer Antibody | R&Dsystems | R&Dsystems; Cat# BAF591 |
| Purified anti-mouse | BioLegend | BioLegend Cat# 117302, RRID:AB_313771 |
| CD11c antibody | | |
| Clec4F antibody | R&Dsystems | R&Dsystems; Cat# AF2784 |
| F4/80 antibody | Bio-Rad | Bio-Rad Cat# MCA497, RRID:AB_2098196 |
| Purified anti-mouse | BioLegend | BioLegend Cat# 141702, |
| CD206 | | RRID:AB_10900233 |
| Purified anti-mouse CD43 | BioLegend | BioLegend Cat# 143202, |
| | | RRID:AB_11124103 |
| CD81 antibody | BD | BD Biosciences Cat# 559518, |
| | Biosciences | RRID:AB_397259 |
| CD226 (DNAM-1) | Thermo | Thermo Fisher Scientific Cat# 16-2261-81, |
| Monoclonal Antibody | Fisher | RRID:AB_1724031 |
| | Scientific | |
| Purified anti-mouse | BioLegend | BioLegend Cat# 121402, RRID:AB_535945 |
| CD103 antibody | | |
| Purified anti-mouse CD64 | BioLegend | BioLegend Cat# 139302, |
| (FcgammaRI) antibody | | RRID:AB_10613107 |
| Purified anti-mouse | BioLegend | BioLegend Cat# 142402, |
| CD169 (Siglec-1) | | RRID:AB_10916523 |
| antibody, | | |
| anti-CCR2 antibody | R&Dsystems | R and D Systems Cat# MAB55381-100, |
| | | RRID:AB_2749828 |
| LYVE1 Monoclonal | Thermo | Thermo Fisher Scientific Cat# 14-0443-37, |
| Antibody | Fisher | RRID:AB_2864884 |
| | Scientific | |
| CD102 (ICAM-2) | Thermo | Thermo Fisher Scientific Cat# 16-1021-82, |
| Monoclonal Antibody | Fisher | RRID:AB_2573077 |
| | Scientific | |
| Purified anti-mouse | BioLegend | BioLegend Cat# 121602, RRID:AB_572021 |
| CD107a (LAMP-1) | | |
| antibody | | |
| Purified anti-mouse Siglec | BioLegend | BioLegend Cat# 129602, RRID:AB_1227757 |
| H antibody | | |
| CD172a antibody | BD | BD Biosciences Cat# 552371, |
| | Biosciences | RRID:AB_394371 |
| Purified anti-mouse CD24 | BioLegend | BioLegend Cat# 101802, RRID:AB_312835 |
| antibody | | |
| Purified anti-mouse | BioLegend | BioLegend Cat# 123902, RRID:AB_1227747 |
| CD200R (OX2R) antibody | | |
| InVivoMab anti-mouse | Bio X Cell | Bio X Cell Cat# BE0212, RRID:AB_2687698 |
| Thy1 (CD90) | | |
| Monoclonal Anti-HA | Sigma- | Sigma-Aldrich Cat# H9658, |
| antibody | Aldrich | RRID:AB_260092 |
| lgG1 Isotype Control | Sigma- | Sigma-Aldrich Cat# M5284, |
| | Aldrich | RRID:AB_1163685 |
| Alexa Fluor ® 488 anti- | BioLegend | BioLegend Cat# 123120, RRID:AB_893479 |
| mouse F4/80 antibody | | |
| APC anti-mouse CD206 | BioLegend | BioLegend Cat# 141708, |
| (MMR) antibody | | RRID:AB_10900231 |
| Alexa Fluor ® 594 anti- | BioLegend | BioLegend Cat# 102725, RRID:AB_2566435 |
| mouse CD38 antibody | | |

Chemicals, Peptides, and Recombinant Proteins

| | | |
|---|---|---|
| Lysyl EndopeptidaseR | Wako | Wako |
| (Lys-C) | | Cat# 129-02541 |
| Collagenase type IV | Sigma | Sigma |
| | | Cat #C5138 |
| DNAsel | Thermo | Thermo Fisher Scientific |
| | Fisher | Cat# EN0521 |
| | Scientific | |
| Sequencing Grade | Promega | Promega |
| Modified Trypsin | | Cat# V5117 |
| RNasin ® Ribonuclease | Promega | Promega |
| Inhibitor | | Cat# N2511 |
| Cycloheximide | Sigma | Sigma |
| | | Cat# 66-81-9 |
| Dynabeads ™ Protein G | Thermo | Thermo Fisher Scientific Cat# 10003D |
| | Fisher | |
| | Scientific | |
| O.C.T. | Killik Bio- | Killik Bio-Optica |
| | Optica | Cat# 05-9801 |
| DAPI | Sigma | Sigma |
| | | Cat# 28718-90-3 |
| FluorSave ™ Reagent | Millipore | Millipore |
| | | Cat# 34578 |

Critical Commercial Assays

| | | |
|---|---|---|
| Pierce ™ BCA Protein | Thermo | Thermo Fisher Scientific Cat# 23227 |
| Assay Kit | Fisher | |
| | Scientific | |
| Amplex ™ Red Hydrogen | Thermo | Thermo Fisher Scientific Cat# A22188 |
| Peroxide/Peroxidase | Fisher | |
| Assay Kit | Scientific | |
| Lipid Peroxidation (MDA) | Abcam | Abcam Cat# ab118970 |
| Assay Kit | | |
| (Colorimetric/Fluorometric) | | |
| Arcturus PicoPure ® RNA | Thermo | Thermo Fisher Scientific Cat# KIT0204 |
| Isolation kit | Fisher | |
| | Scientific | |
| DNA High Sensitivity | Perkin | Perkin Elmer |
| Reagent Kit | Elmer | Cat# CLS760672 |
| Illumina Nextera XT kit | Illumina | Illumina |
| | | Cat# FC-131-1024 |

Deposited Data

| | | |
|---|---|---|
| Single-cell RNaseq data | This | NCBI Gene Expression Omnibus(GEO) |
| | manuscript | accession #168989 |

Experimental Models: Organisms/Strains

| | | |
|---|---|---|
| Mouse: C57BL/6 | The | C57BL/6 colony |
| | Jackson | |
| | Laboratory | |
| Oligonucleotides | | |
| 5′- | Dharmacon | N/A |
| GCAAAUGCAAAGAAGG | | |
| AAA-3′ (SEQ ID NO: 1) | | |
| 5′- | Dharmacon | N/A |
| CAGUCGCGUUUGCGAC | | |
| UGG-3′ (SEQ ID NO: 2) | | |

Software and Algorithms

| | | |
|---|---|---|
| FlowJo V10 | FlowJo | https://www.flowjo.com/ |
| R v4.4 | The R | https://www.r-project.org |
| | Foundation | |
| CytofKit | Chen et al., | https://bioconductor.riken.jp/packages/ |
| | 2016 | 3.3/bioc/html/cytofkit.html |
| Seurat v2 | Butler et al., | https://satijalab.org/seurat/ |
| | 2018 | |
| Seurat v3 | Stuart et al., | https://satijalab.org/seurat/ |
| | 2019 | |
| One sense | Cheng et | N/A |
| | al., 2016 | |
| MaxQuant 1.6.7.0 | MaxQuant | https://www.maxquant.org |
| Subread package | N/A | http://subread.sourceforge.net |
| edgeR | Robinson et | http://bioconductor.org/packages/release/ |
| | al., 2010 | bioc/html/edgeR.html |
| LIMMA R package | Ritchie et | https://bioconductor.org/packages/release/ |
| | al., 2015 | bioc/html/limma.html |
| COMBAT | Wang et al., | https://cran.r-project.org/web/packages/ |
| | 2017 | COMBAT/index.html |
| Short Time-series | N/A | https://www.cs.cmu.edu/~jernst/stem/ |
| Expression Miner (STEM) | | |
| Imaris bitplane | Imaris | https://imaris.oxinst.com/products/ |
| | | imaris-for-cell- |
| | | biologists?gclid=Cj0KCQiAgomBBhDXARIsA |
| | | FNyUqOQMD64vZvZMyBoHWFOYRm_ZPxHWLb_ |
| | | tWDI0pGjii8ZVNDkW- |
| | | UNtRgaAnhfEALw_wcB |

Other

| | | |
|---|---|---|
| LSRII | BD | N/A |
| | Biosciences | |
| Imagestream Amnis | Merk | N/A |
| FACSAria II | BD | N/A |
| | Bioscience | |
| FACSAria III | BD | N/A |
| | Bioscience | |
| Mindray BS-240 Pro | BioSentec | N/A |
| Easy LC 1000 | Thermo | N/A |
| | Scientific | |
| Obritrap Fusion Lumos | Thermo | N/A |
| | Scientific | |
| Agilent Bioanalyser | Agilent | N/A |
| Perkin Elmer Labchip | Perkin | N/A |
| | Elmer | |
| Illumina HiSeq 4000 | Illumina | N/A |
| system | | |
| Chromium Single Cell 3′ | 10× | N/A |
| (v3 Chemistry) | Genomics | |
| SP5 confocal microscope | Leica | N/A |
| | Microsystem | |
| JSM-6701F scanning | JEOL Ltd. | N/A |
| electron microscope | | |

### Results

### Unbiased Approaches Reveal KC Transcriptomic Heterogeneity

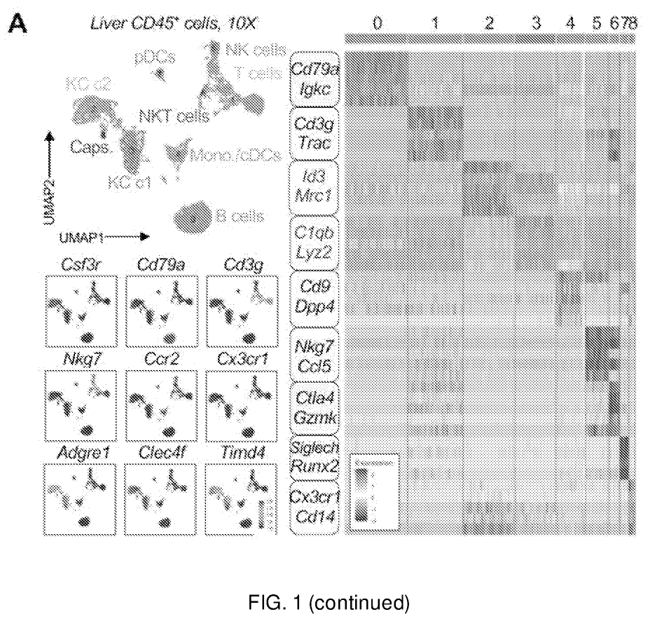

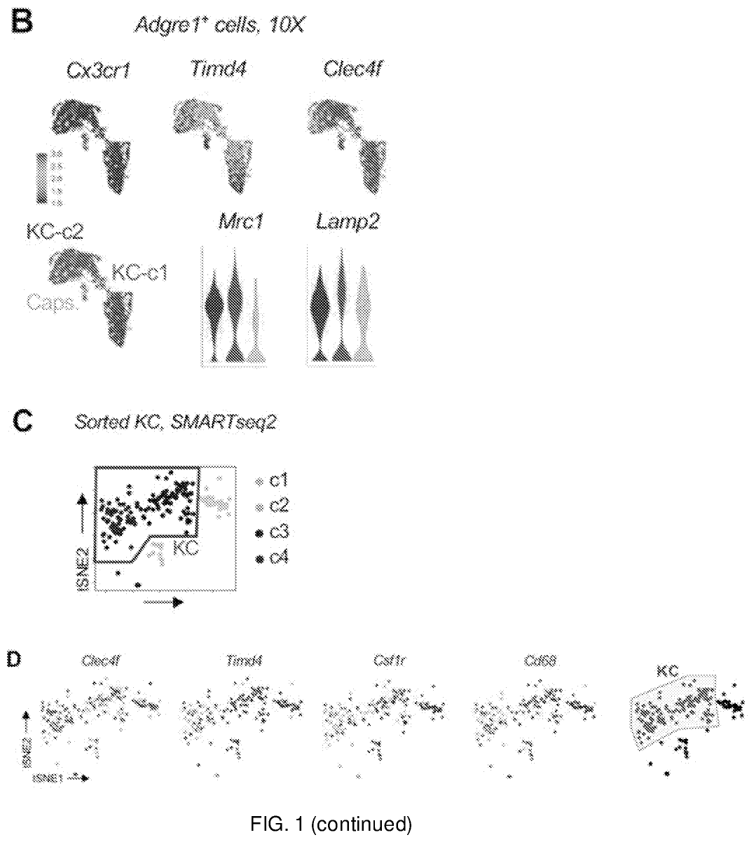

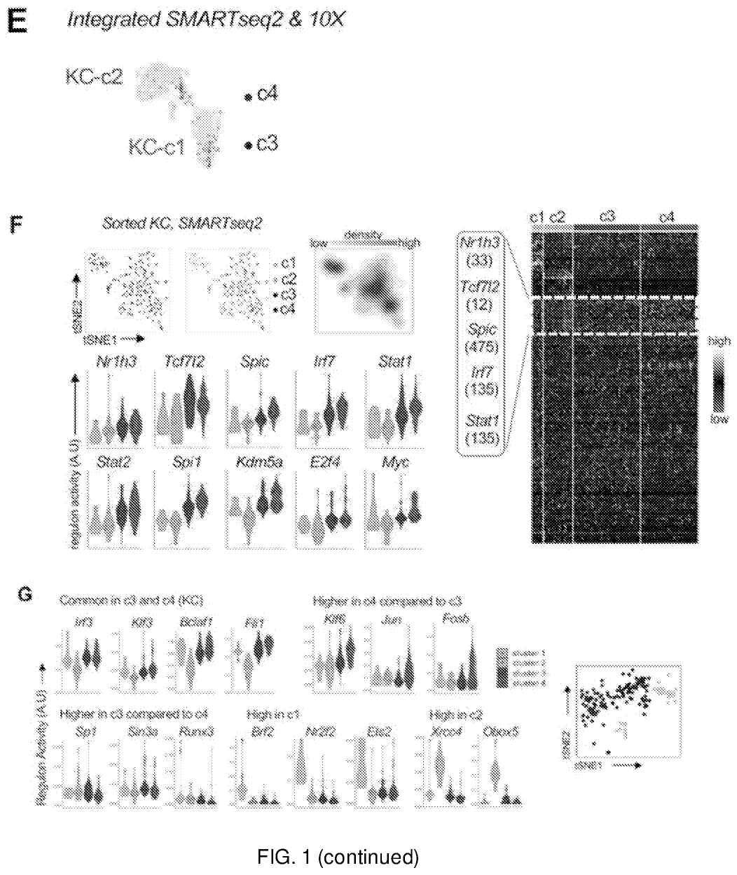

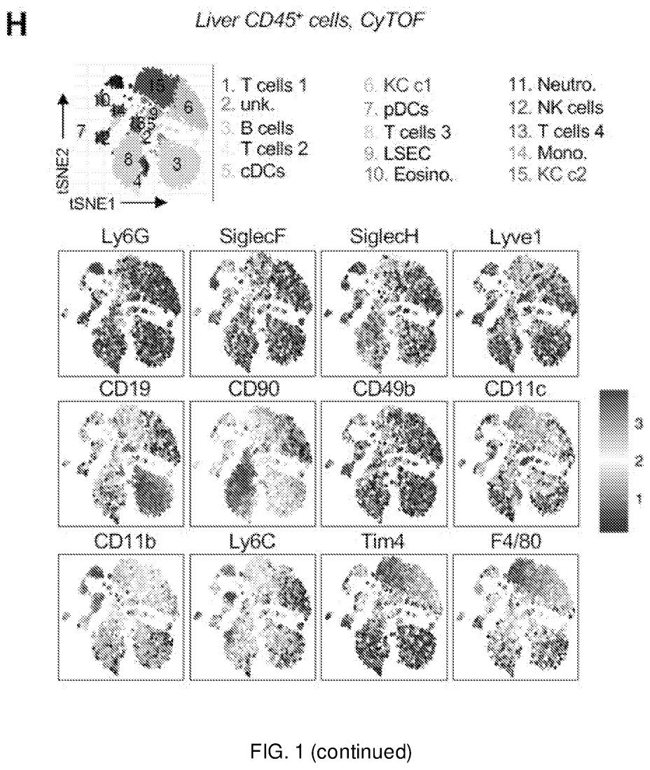

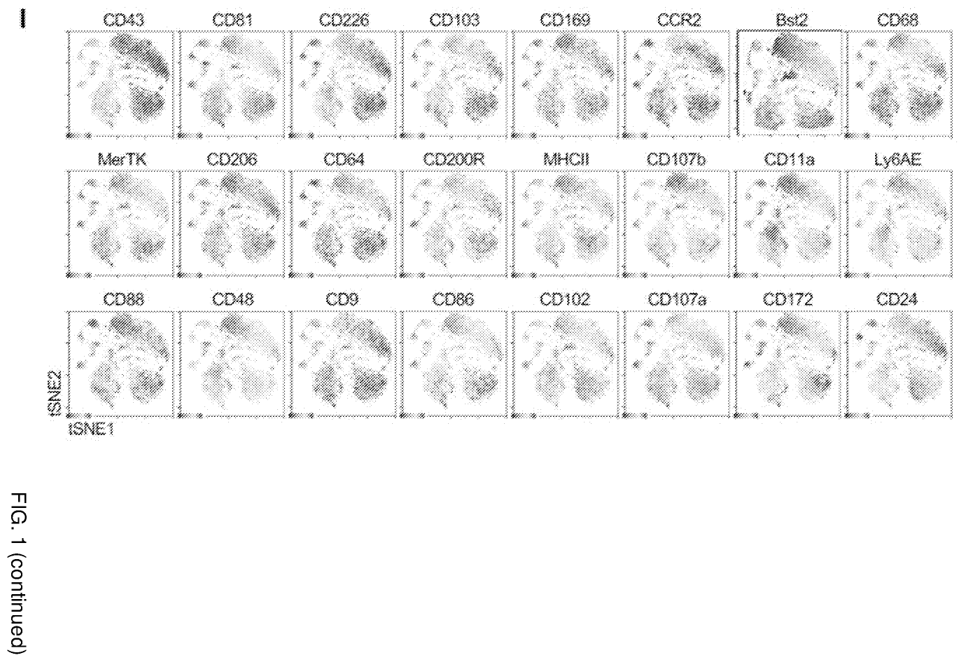

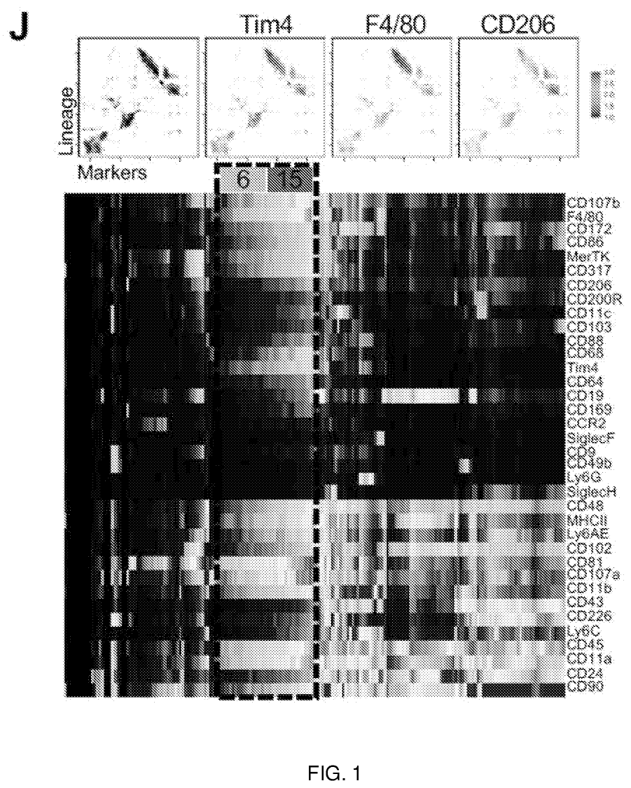

To assess the KC heterogeneity in an unbiased manner, the inventors of the present disclosure first employed single cell RNA-seq technology. FIG. 1A to 1G shows unbiased approaches that reveal KC heterogeneity. The inventors of the present disclosure purified liver CD45+ leukocytes from steady state murine liver and profiled thousands of individual cells by using the Chromium single cell gene expression technology (10×). A Seurat analysis of this dataset with a uniform manifold approximation and projection (UMAP) dimension reduction analysis and an automatic clustering identified 9 main clusters (#0 to #8) in the CD45+ cell population (FIG. 1A). These clusters were manually annotated by using canonical population markers: #0 represents B cells, #1 T cells, #4 monocytes and cDCs, #5 NK cells, #6 NKT cells, #7 pDCs and #8 capsular macrophages.

Concerning clusters #2 and #3, these cells co-expressed many genes being classically highly expressed by Kupffer cells (KCs) such as Adgre1 (encoding F4/80), Timd4, Csf1r and Clec4f (FIG. 1A). When focusing on total Adgre1+ cells, a population that can be considered as liver macrophages, the inventors of the present disclosure identified a sub-cluster of Cx3cr1+Timd4− Clec4f− cells corresponding to capsular macrophages, and two clusters of Cx3cr1− Timd4+Clec4f+ KCs expressing differentially genes such as Mrc1 (CD206) and Lamp2 (CD107b) (FIG. 1B).

In addition, using the high resolution SMARTseq2 platform, the inventors of the present disclosure have observed that sorted liver CD64+F4/80+ cells could be split into four clusters (FIG. 1C). From these clusters, only the two main ones c3 and c4 harbored the canonical KC signature while c1 and c2 displayed unrelated signatures (FIG. 1D), arguing from a contamination during cell sorting. Then, by integrating the low resolution 10× data generated on thousands of cells with the deeper but number-restricted SMARTseq2 data, the inventors of the present disclosure validated the presence of two clusters of KCs observed with two different single cell RNA-seq technologies (FIG. 1E).

The inventors of the present disclosure then used the Scenic analytical pipeline to reveal regulatory networks in the most sensitive SMARTseq2 single cell RNA-seq dataset. This confirmed that the full KC population harboring a pattern of regulon activity consistent with liver macrophages, with a high expression of canonical KC transcription factors such as Nr1h3 (liver X receptor alpha) and Spic, could be split into two different states (FIG. 1F).

Moreover, each cluster of KCs displayed a specific regulon activity profile, for example with Runx3 being more active in c3 and Klf6 more active in c4 (FIG. 1G). The unbiased single cell RNA-seq approaches in the current invention revealed therefore two distinct subsets of KCs present at steady-state. To validate these findings at the protein level, the inventors of the present disclosure used the mass cytometry technology to monitor the expression of an extended panel of common myeloid markers and putative markers identified by the unbiased single cell transcriptomic approach, such as CD206 and CD107b (FIG. 1H & 1I). By integrating the expression of these markers in live CD45+ cells from steady state murine liver, the inventors of the present disclosure identified the major immune cell subsets present in the liver: CD19+B cells (#3), CD90+ T cells (#1, #4, #8 & #13), CD49b+ NK cells (#12), SiglecH+BST2+ pDCs (#7), Ly6C+ monocytes (#14), CD11c+ cDCs (#5), SiglecF+ eosinophils (#10), Ly6G+ neutrophils (#11) and a large population of F4/80+Tim4+ KCs comprised of 2 clusters (#6 & #15) (FIG. 1H & 1I).

Focusing on this KC population, a One-SENSE (one-dimensional soli-expression by nonlinear stochastic embedding) analysis allowing the manual definition of lineage and marker dimensions revealed some markers differentially expressed by the two clusters within this population, notably CD206 but also CD107a & b, CD81 and Lyve1 (FIG. 1J).

Therefore, by combining unbiased transcriptomic and proteomic approaches, the inventors of the present disclosure uncovered that KC can be subdivided in CD206lo CD107b−(=KC1) and CD206hi CD107b+ (=KC2) populations.

KCs can be Divided into Two Subpopulations Sharing a Common Embryonic Origin

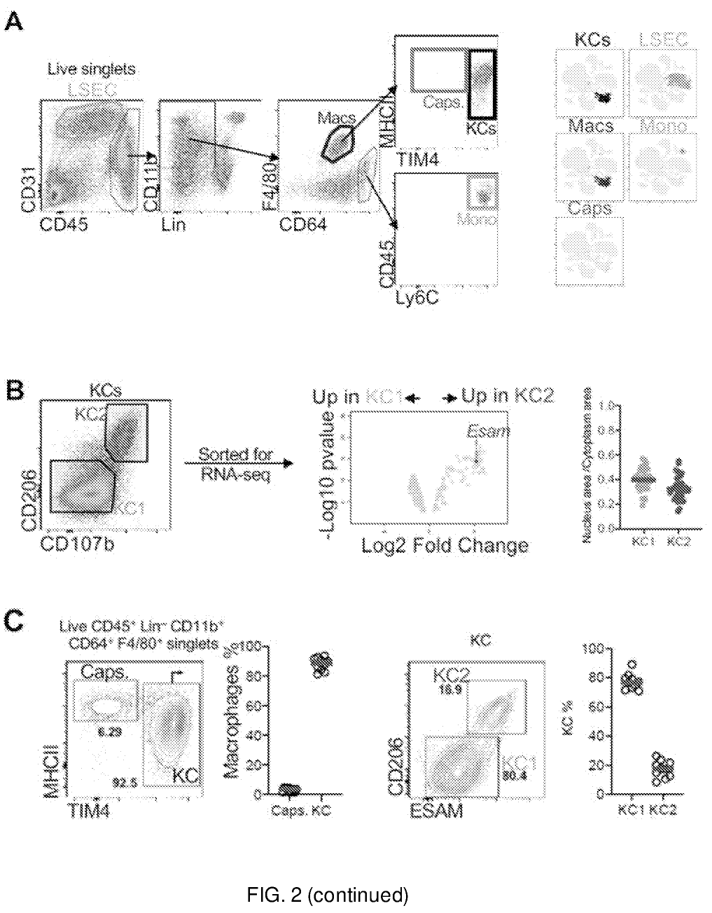

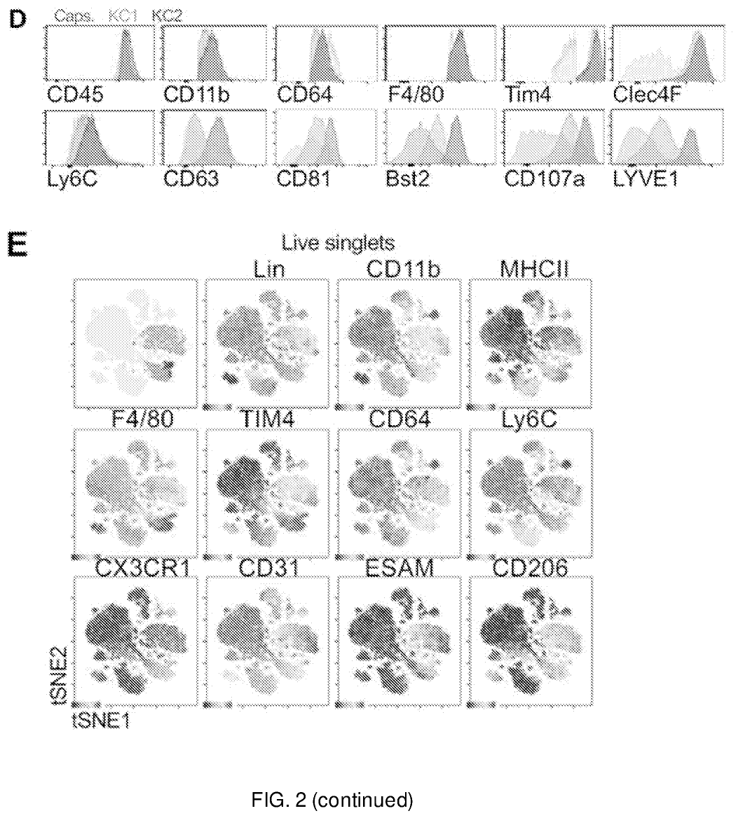

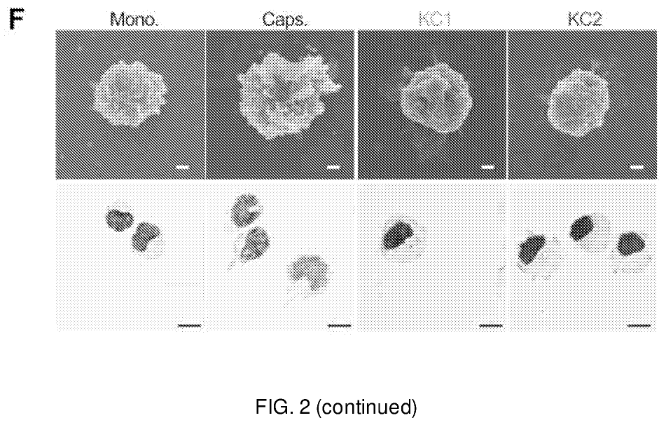

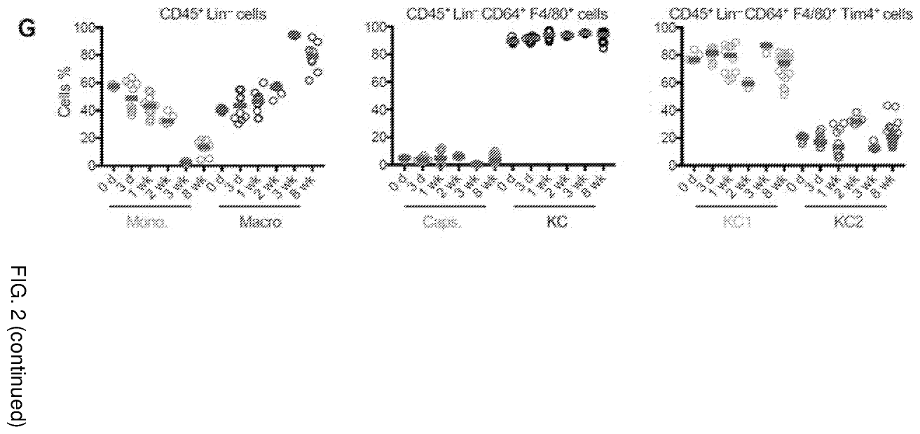

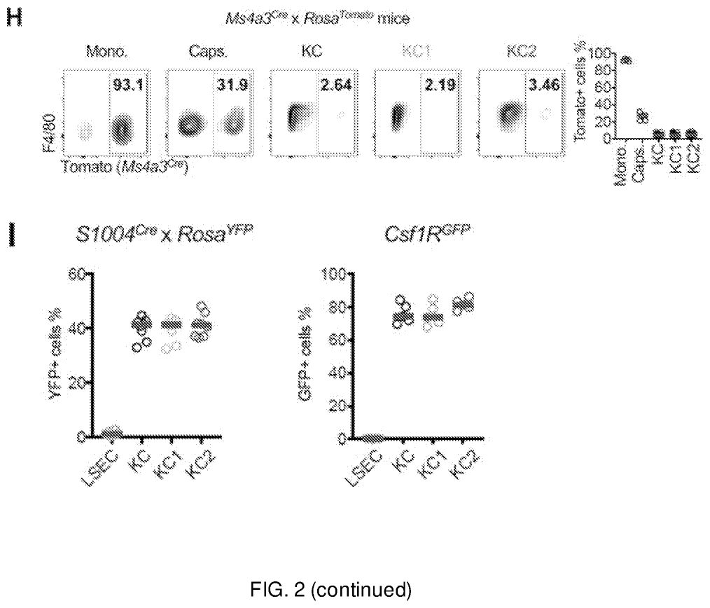

Based on this unsupervised approach, the inventors of the present disclosure designed a panel of markers for use in conventional flow cytometry to analyse the KC population. KCs are classically defined as CD45+CD64+F4/80+TIM4+Clec4F+ cells with low expression of CD11b and Ly6C (FIG. 2A). The inventors of the present disclosure used CD206 and CD107b to identify and sort KC1 and KC2 and generate transcriptomic signatures of these two populations (FIG. 2B).

Among the top differentially expressed genes (DEGs) is Esam, a previously reported marker for splenic dendritic cells. Therefore, the inventors of the present disclosure retained CD206 and ESAM as two reliable markers and used these markers later on to define CD206lo ESAM− KC1 and CD206hi ESAM+ KC2 by conventional flow cytometry (FIG. 2C). The inventors of the present disclosure also measured expression of other markers highlighted as over-expressed by KC2 with high-throughput approaches including CD63, CD81, CD107a and Lyve1 on the two populations (FIG. 2D).

All these markers were expressed by KC2, and the inventors of the present disclosure could not identify any specific markers of KC1. However, KC1 and KC2 could be clearly distinguished either by manual gating based on CD206 and ESAM expression or by using algorithm-based dimension reduction (FIG. 2E). Sorted KC1 and KC2 had comparable morphology and were indistinguishable by electronic microscopy and cytospin (FIG. 2F).

The inventors of the present disclosure and other authors of studies known in the art have shown that KCs are derived from fetal liver monocytes with a minimal contribution of bone marrow monocytes to maintain the adult KC population. However, several reports have shown that monocyte-derived cells could acquire KC identity in non-homeostatic conditions. Thus, to clarify the origin of the two subpopulations and accurately categorize them as bona fide KCs, the inventors of the present disclosure first checked when heterogeneity within the KC population emerged and analyzed the presence of KC1 and KC2 from birth to adulthood. While the ratio of monocytes to macrophages was dynamic during the first postnatal weeks, the ratio between KC1 and KC2 populations was very stable, being already established at birth, suggesting that they arise both from prenatal precursors (FIG. 2G), even if the inventors of the present disclosure could not formally exclude that each subpopulation emerged during distinct waves in embryogenesis.

The inventors of the present disclosure then used the monocyte fate-mapping Ms4a3^{cre}xRosa^{Tomato} mouse model to assess any possible monocytic contribution. As expected, monocytes and monocyte-derived capsular macrophages were highly tagged in adult mice, whereas KC1 and KC2 exhibited an equal and very low tagging (FIG. 2H). In addition, high and comparable reporter expression were observed in KC1 and KC2 in S100a4^{Cre}xRosa^{YFP} and Csf1R^{GFP} mice confirming the macrophage nature of both subpopulations (FIG. 2I).

The inventors of the present invention have also confirmed this by analyzing CD45.1/CD45.2 parabiotic mice in which the non-host chimerism was very low after 3 months of shared systemic circulation in both KC1 and KC2, in contrast to monocyte-derived capsular macrophages (FIG. 2J), as previously shown by other studies known in the art.

Taken together, these results show that both KC subsets are bona fide embryonically derived KCs, therefore do not represent ontogenically distinct populations but rather two different states of KCs.

KC1 and KC2 have Overlapping Distribution Patterns In Situ

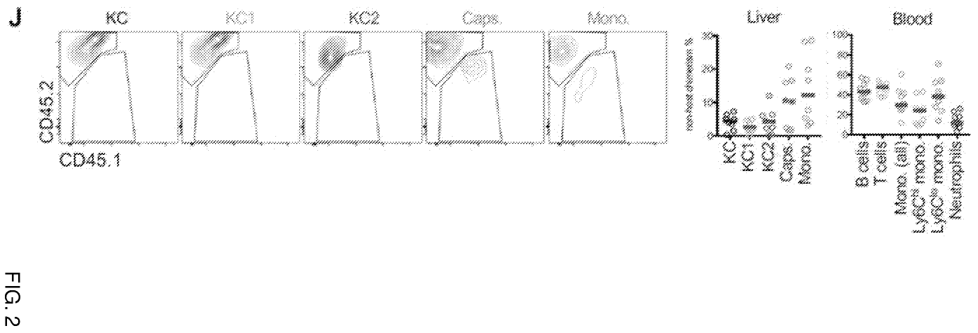

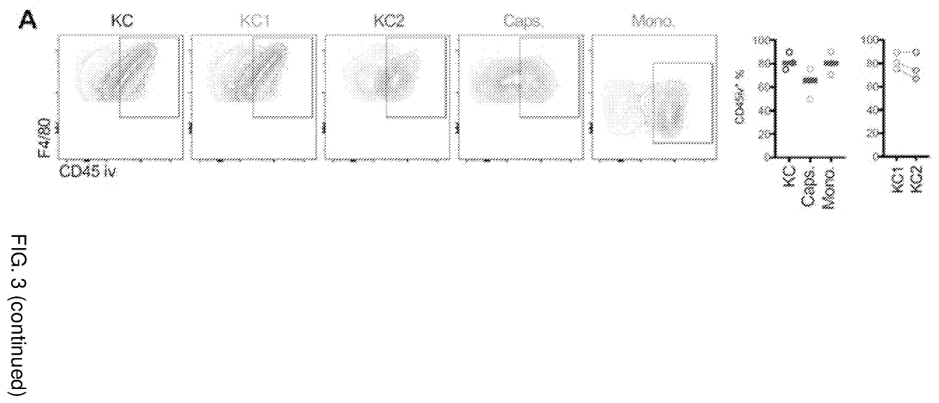

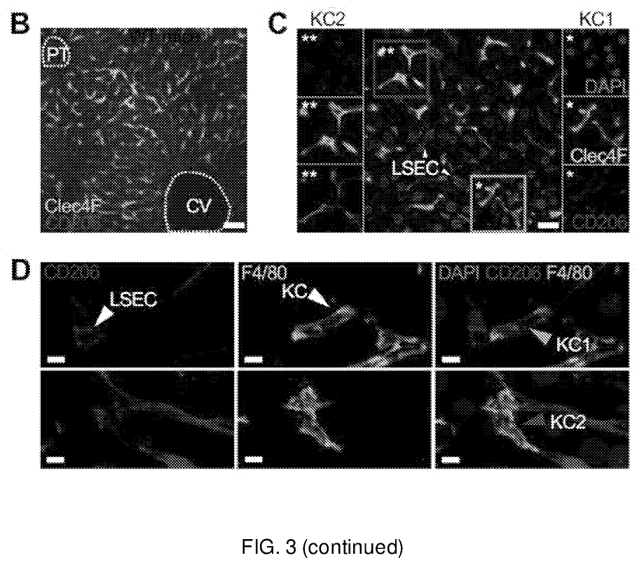

To assess a potential differential sublocalization of the two subsets in the liver, the inventors of the present disclosure first checked their accessibility to an intravenously injected anti-CD45 antibody. KC1 and KC2 were labelled with a comparable efficiency confirming their sinusoidal localization (FIG. 3A). The inventors of the present disclosure then used two-photon microscopy to look at these cells in situ coupled with immunofluorescence microscopy of liver sections from WT mice for more detailed analysis. Clec4F+ KCs were easily distinguished from the Clec4F−CD206+ LSEC and KC2 were detected as Clec4F+ or F4/80+ KCs also expressing CD206 (FIG. 3B-D).

To fully validate this, the inventors of the present disclosure generated Mrc1cre^{ERT2} mice (FIG. 3E) and bred them with Rosa^{Tomato} mice in order to establish a model in which the inventors of the present disclosure can track cells expressing CD206 after tamoxifen induction. By immunostaining, the inventors of the present disclosure observed that around 15% of KC were tagged with tomato in these mice, in accordance with the proportions observed by using the CD206 marker in conventional flow cytometry analysis (FIG. 3F).

The inventors of the present disclosure also measured the distance between F4/80+CD206lo KC1 and F4/80+CD206hi KC2 and their closest portal triad to assess whether hepatic metabolic zonation influenced KC1 and KC2 distribution. No significant differences were observed concerning the zonation of the two populations (FIG. 3G). Taken together, these observations suggest that KC1 and KC2 have overlapping distribution patterns in the liver.

KC2 Express LSEC-Associated/Endothelial Markers but are Distinct from LSEC

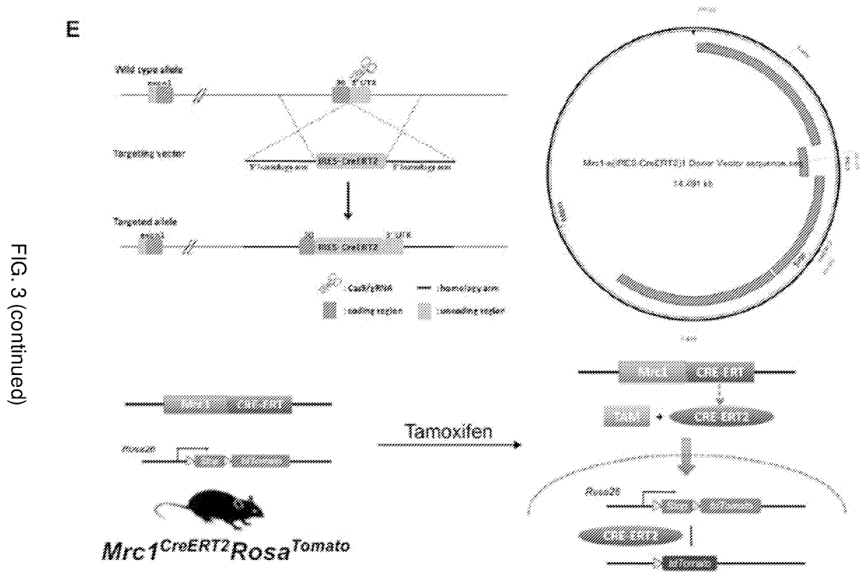

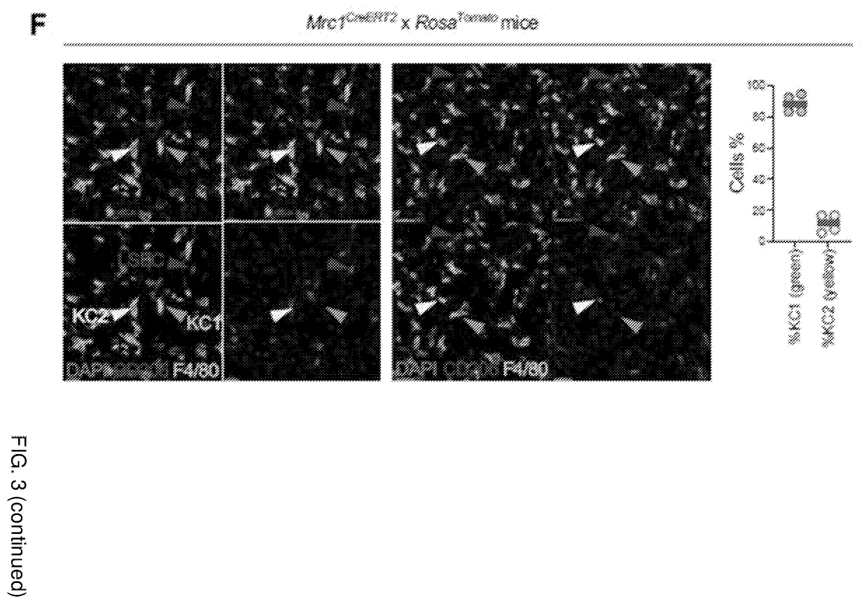

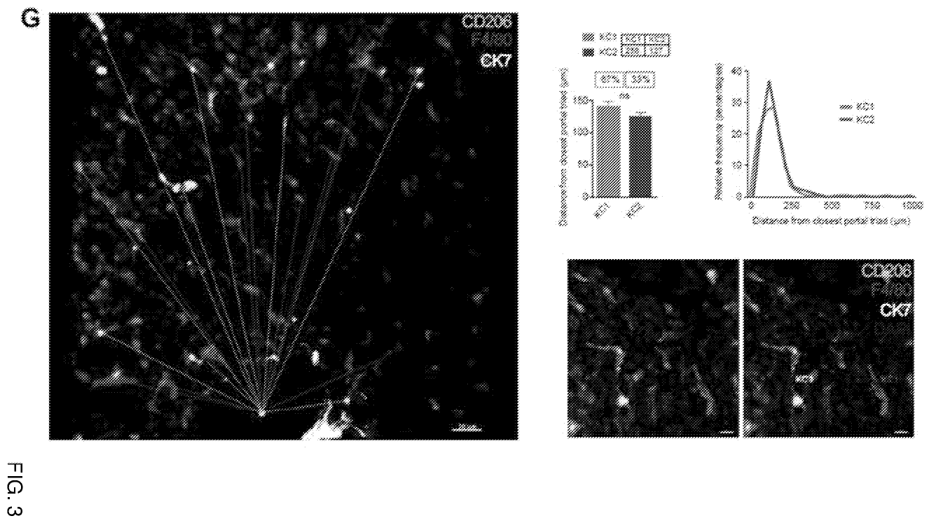

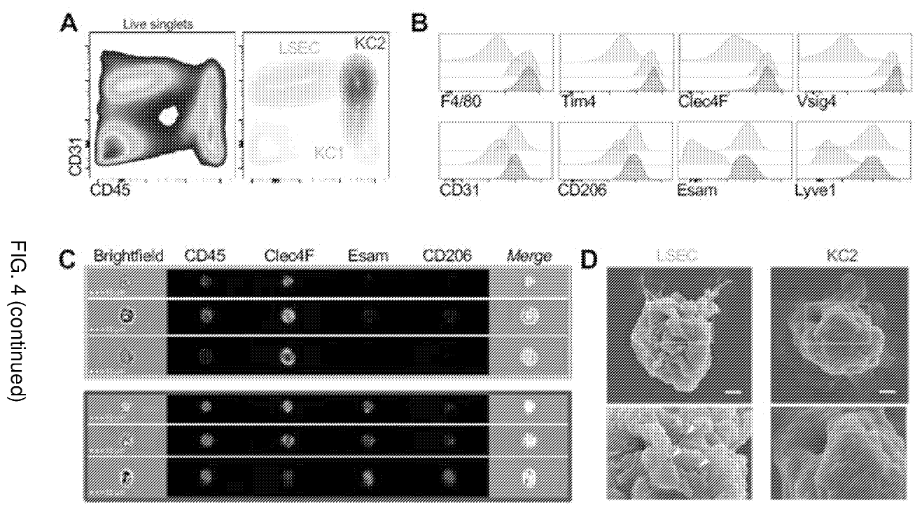

Being macrophages, KC1 and KC2 express canonical macrophage markers such as F4/80, Tim4, Clec4F and Vsig4. But KC2 also displayed markers known to be expressed by endothelial cells such as CD206, CD31, ESAM and even Lyve1 that the inventors of the present disclosure have recently identified as a quite universal macrophage marker (FIG. 4A & 4B).

To understand such expression pattern and exclude the possibility that their detection arose from phagocytosis of liver sinusoidal endothelial cells (LSEC) by KCs, the inventors of the present disclosure performed an image stream analysis coupling flow cytometry with microscopy, allowing the imaging of individual cells. The inventors of the present disclosure observed that ESAM and CD206 labeling was uniformly distributed across KC2 cells, arguing for surface expression of these markers (FIG. 4C). Furthermore, LSEC have fenestrae on their surface, which were absent on KC2 cells (FIG. 4D).

Moreover, the inventors of the present disclosure analyzed Lyz2^{cre}xRosa^{YFP} mice in which cells expressing the canonical myeloid gene Lyz2 are tagged and observed that KC1 and KC2 were both highly labeled while endothelial cells were not tagged (FIG. 4E). The inventors of the present disclosure also confirmed the macrophage nature of KC1 and KC2 by using the Rhapsody technology which allows rapid parallel sequencing of 400 markers in thousands of single cells. Comparing this lower-resolution single-cell transcriptomic profile of sorted KC1 and KC2 to the whole liver leukocyte population, the inventors of the present disclosure found that both KC1 and KC2 clearly clustered in the macrophage population, further confirming their macrophage identity (FIG. 4F).

At the functional level, the inventors of the present disclosure used the clodronate liposome approach to test the phagocytic capacity of both populations: upon liposome injection, KC1 and KC2, but not LSEC, were similarly depleted, indicating comparable phagocytic activity (FIG. 4G).

In addition, KC depletion is known to induce a recruitment of monocytes that quickly acquire KC-like phenotype even if few key KC genes such as Timd4 are not re-expressed before several weeks. When clodronate liposome-mediated depletion was performed in Ms4a3^{cre}xRosa^{Tomato} mice, the inventors of the present disclosure observed that CD64+Tomato+Tim4− monocyte-derived macrophages quickly gave rise to both CD206lo ESAM− KC1-like and CD206hi ESAM+ KC2-like populations in a ratio comparable to steady-state (FIG. 4H). This underlined the fact that recruited naive adult monocytes have the capacity to acquire both KC1- and KC2-like profiles and suggests that both KC1 and KC2 identities are strongly dictated by the liver microenvironment.

### KC1 and KC2 Exhibit Distinct Gene and Protein Expression Signatures

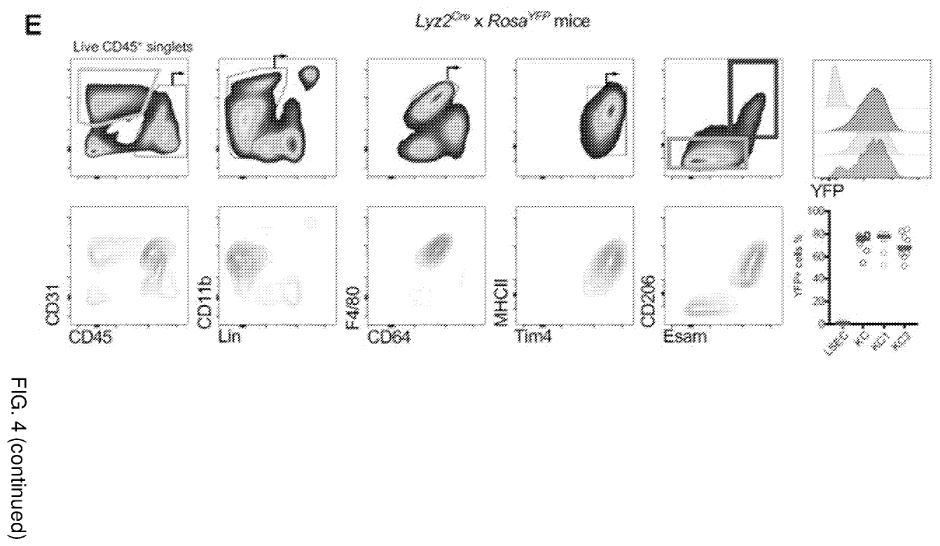

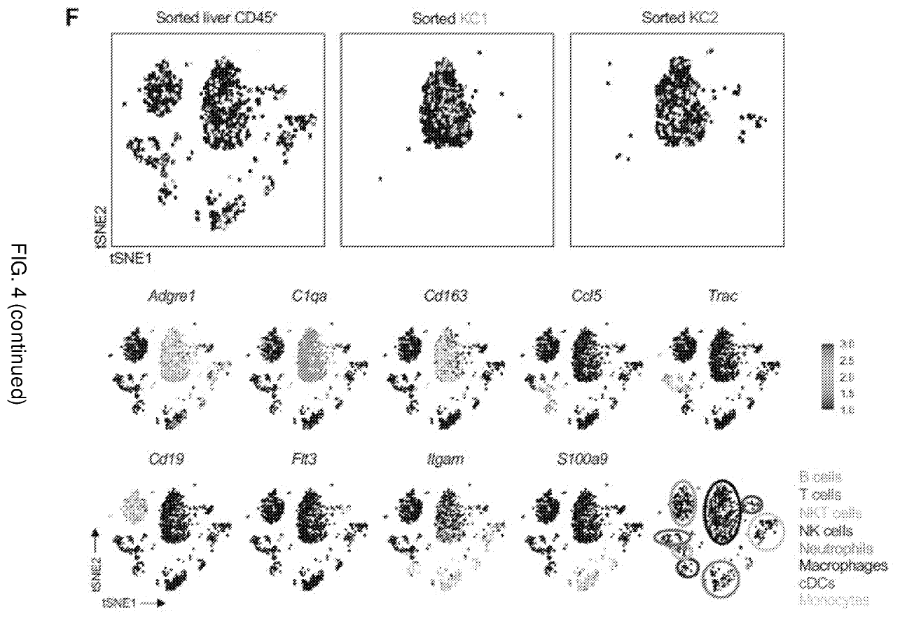

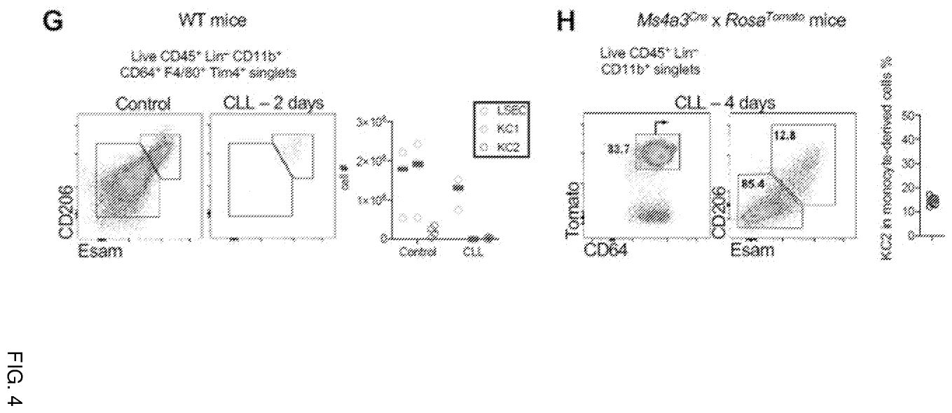

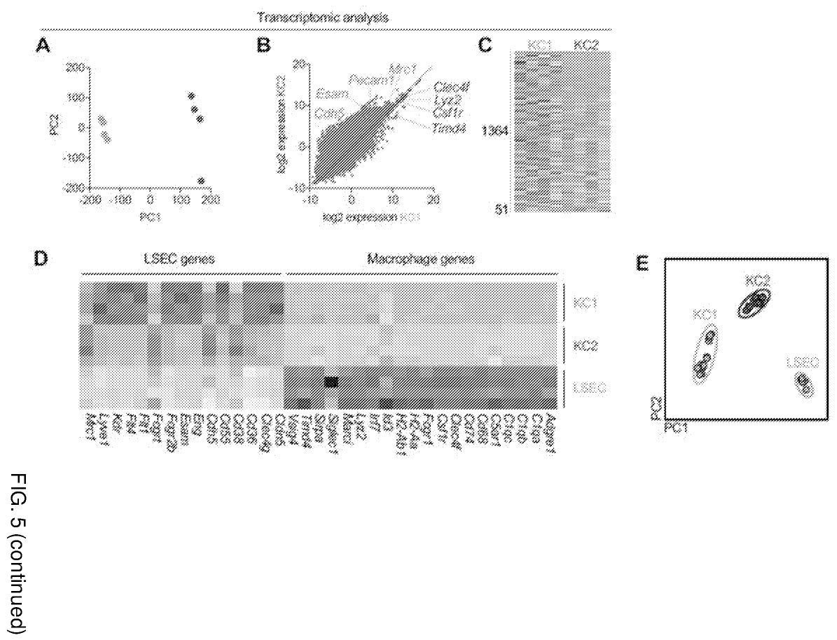

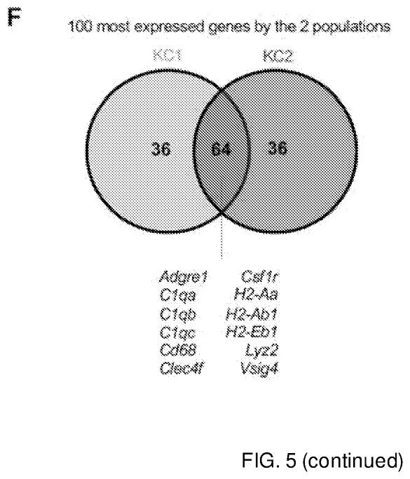

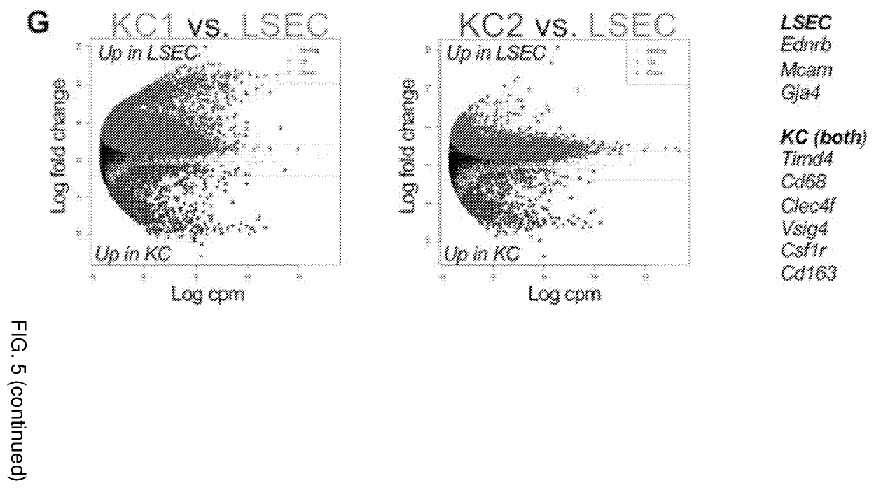

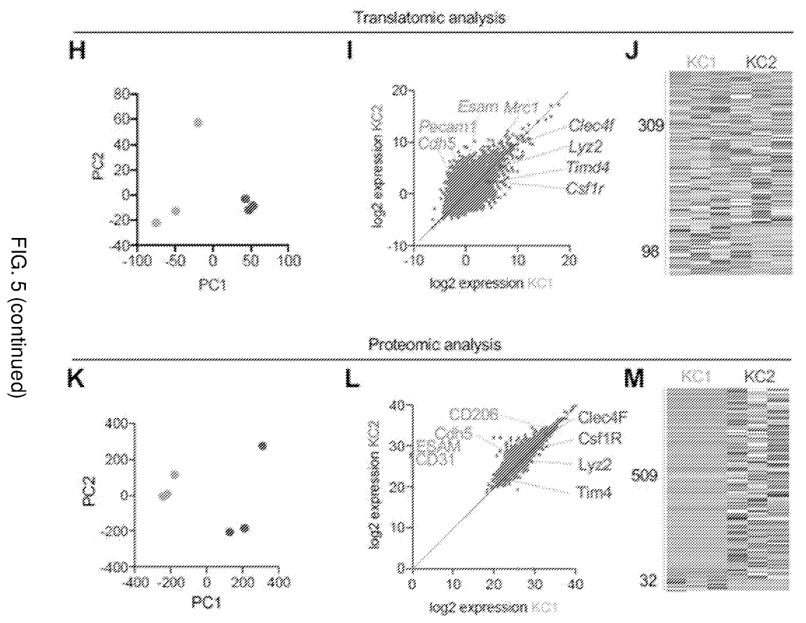

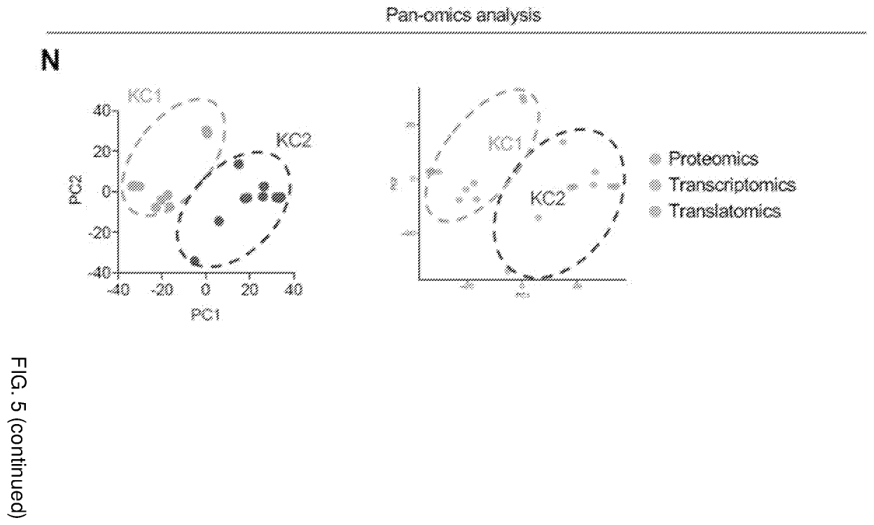

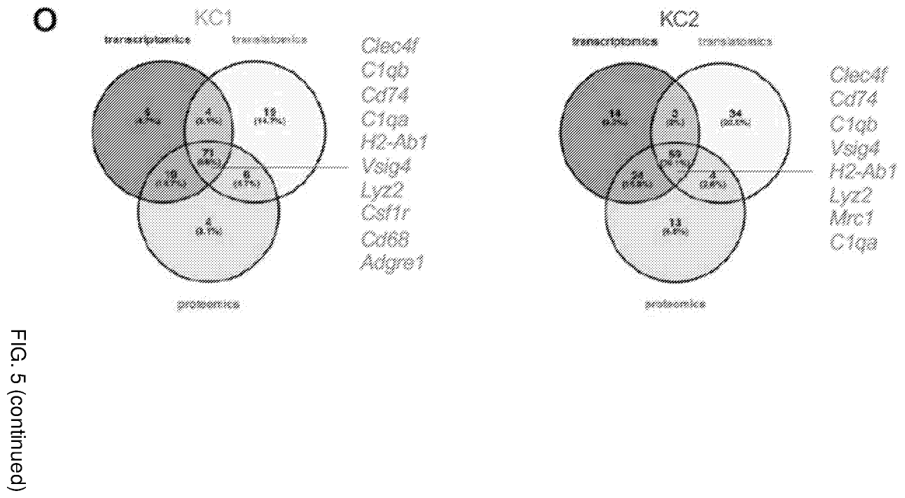

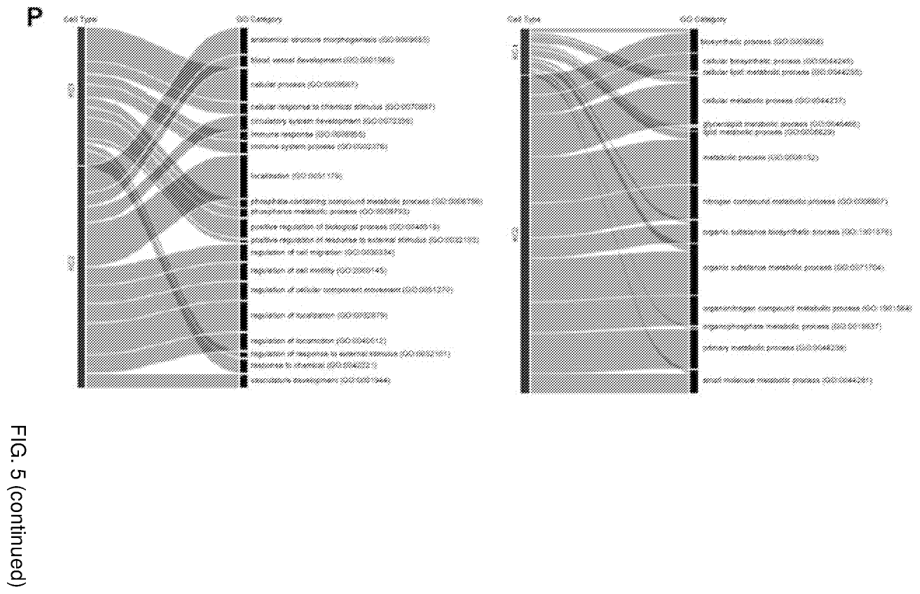

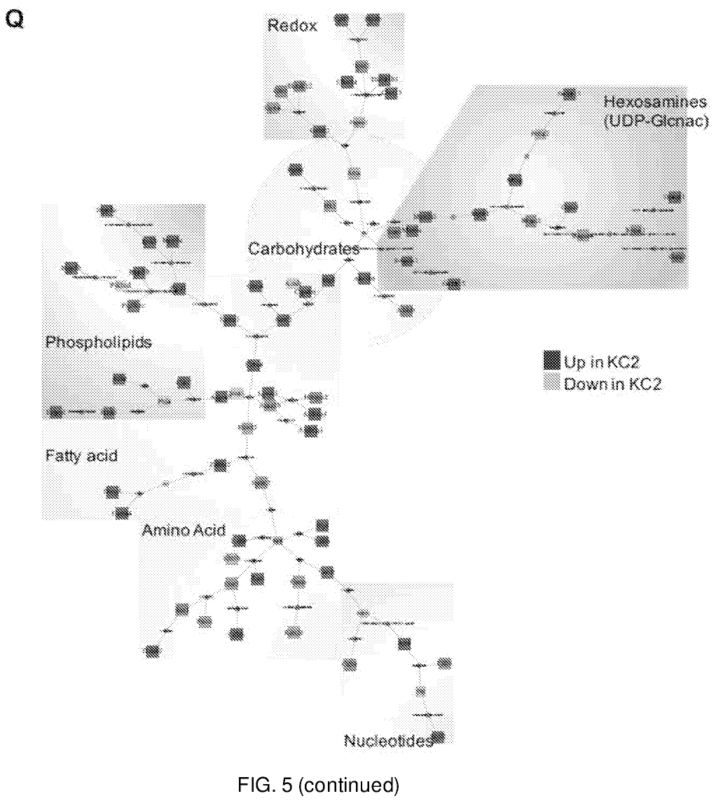

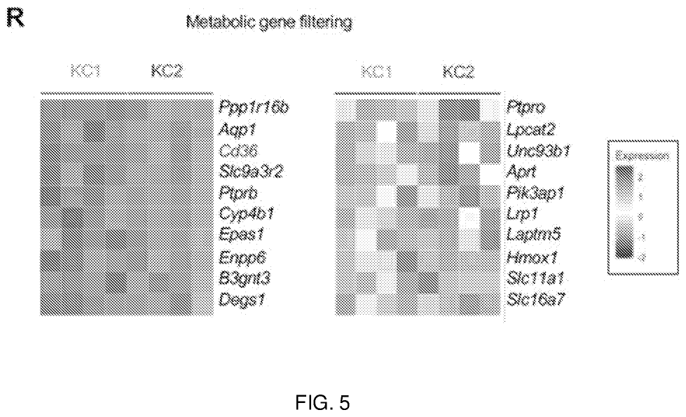

The inventors of the present disclosure then sorted CD206loESAM− KC1 and CD206hiESAM+ KC2 to perform a deeper transcriptomic analysis using the SMARTseq2 protocol for bulk RNA-seq in order to better understand their functions. At this high-resolution, the two populations were clearly separated from each other (FIG. 5A-C), and also distinct from sorted CD45−CD31+ LSEC (FIG. 5D & 5E). KC1 and KC2 expressed similarly canonical macrophage genes including Clec4f, Lyz2, Vsig4, Csf1r and Adgre1 (F4/80) (FIG. 5F & 5G).

In addition to this macrophage signature, as the inventors of the present disclosure found before, KC2 highly expressed numerous LSEC-associated genes, notably Mrc1, Pecam1 (CD31), Esam, Kdr and Lyve1. To confirm that these LSEC-associated genes were indeed expressed by KC2 and were not detected due to phagocytosis of LSEC, the inventors of the present disclosure assessed the actively transcribed RNA (translatome) of the two populations, using the RiboTag strategy. The inventors of the present disclosure generated Lyz2^{cre}×Rpl22^{HA} mice in which the inventors of the present disclosure could purify the ribosomes from both KC1 and KC2, and sequenced the RNA associated with them. Using this method, even though the quantity of the initial material and accordingly the global detected expression decreased, the dichotomy between KC1 and KC2 remained, including KC2 expression of the “LSEC-associated genes” Mrc1, Esam and Lyve1 (FIG. 5H-J).

At the protein level, the inventors of the present disclosure also measured expression of around 4,500 proteins in sorted KC1 and KC2 by mass spectrometry and found comparable differentially expressed proteins, notably ESAM and CD31 that were more abundant in KC2 (FIG. 5K-M). Finally, the inventors of the present disclosure integrated the transcriptome, translatome and proteome data to generate robust KC1 and KC2 pan-omics identities (FIG. 5N): while the core KC program was conserved across both populations (FIG. 5O FIG. 14B), pathway analysis identified a stronger immune signature for KC1, whereas cell adhesion and notably genes related to metabolic pathways notably Cd36 were upregulated in KC2 (FIG. 5P-R).

### KC2 Exhibit Metabolic Functions

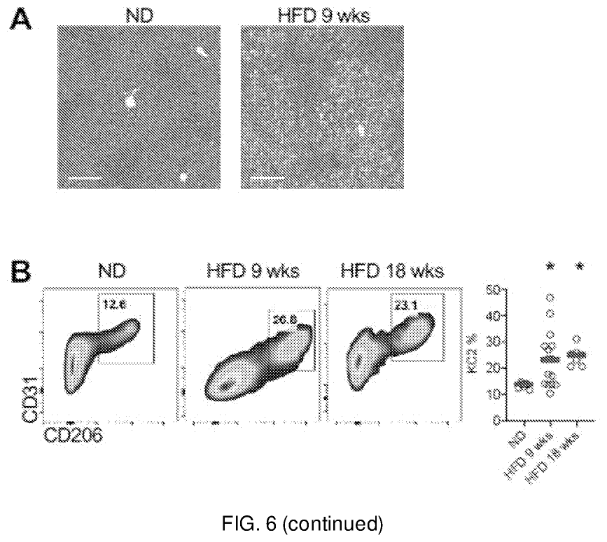

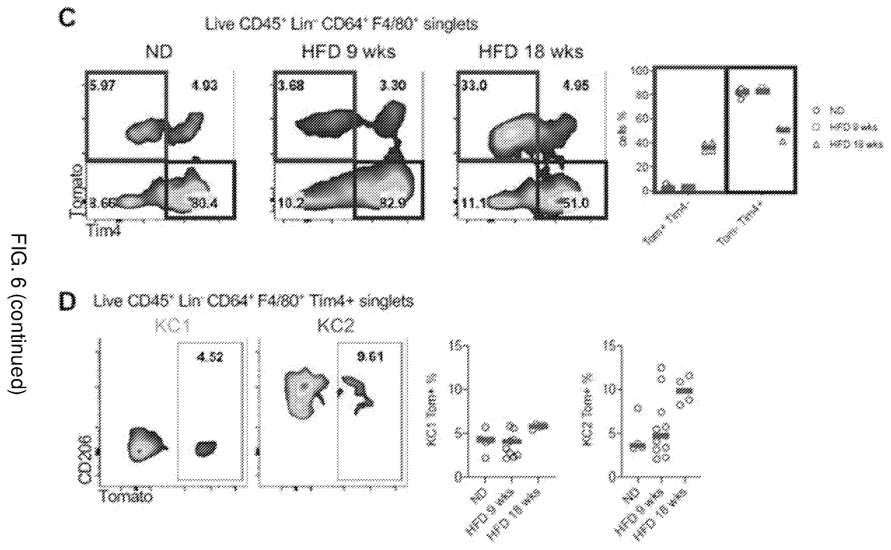

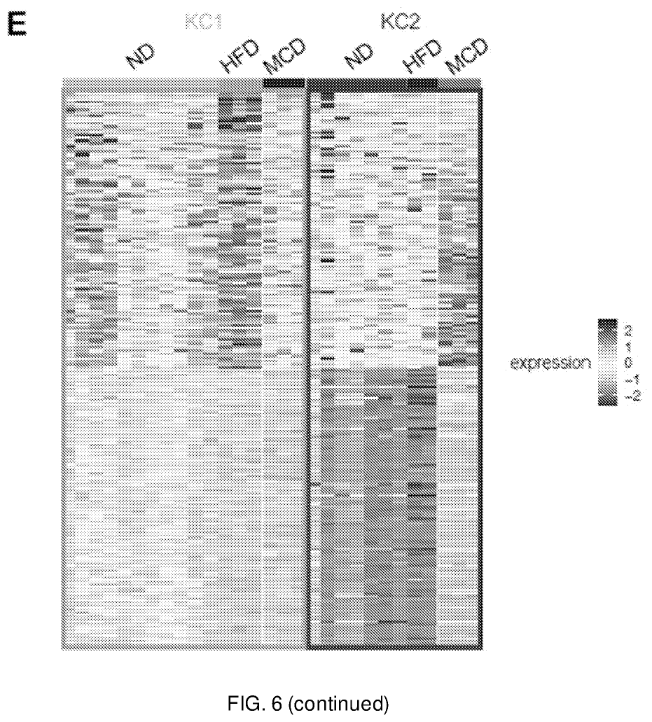

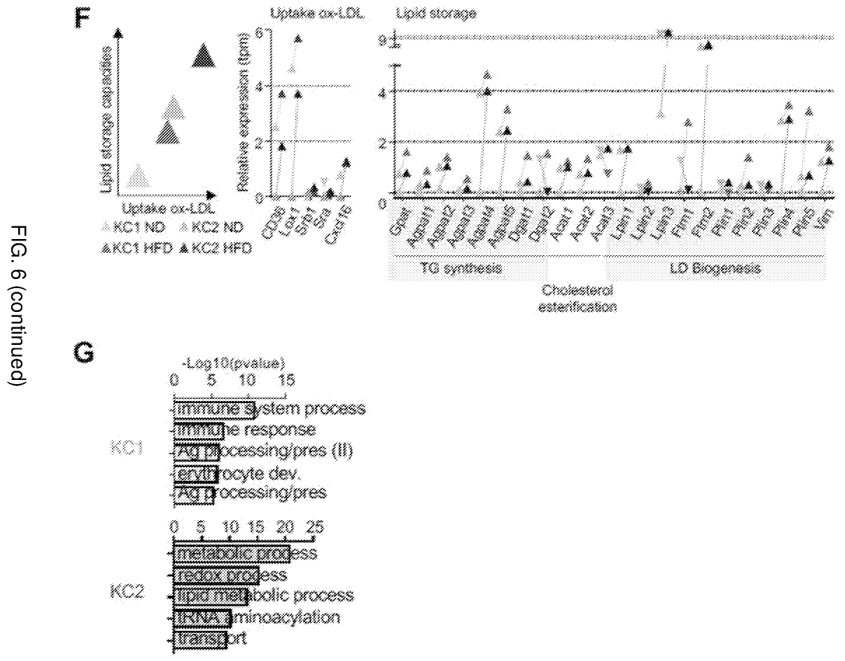

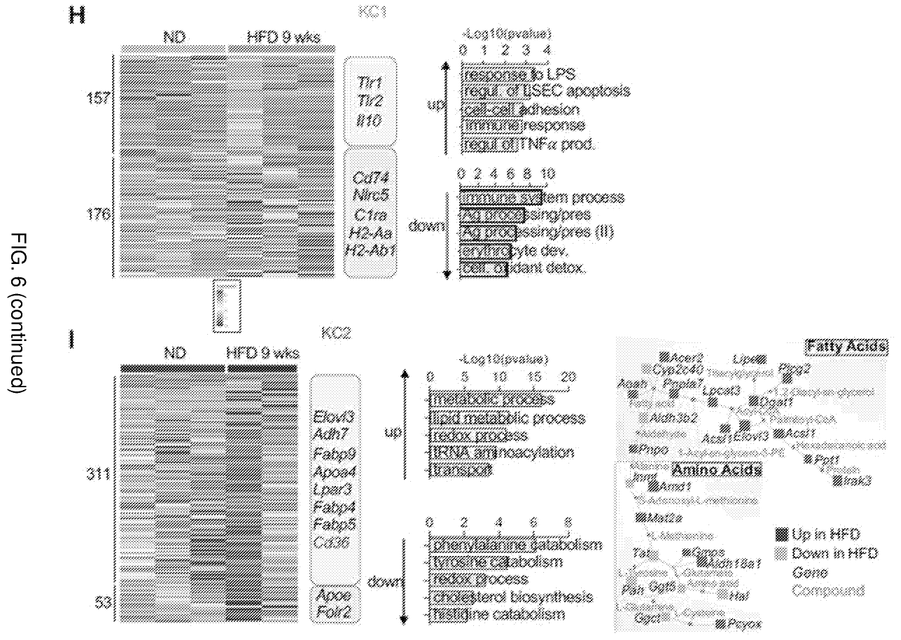

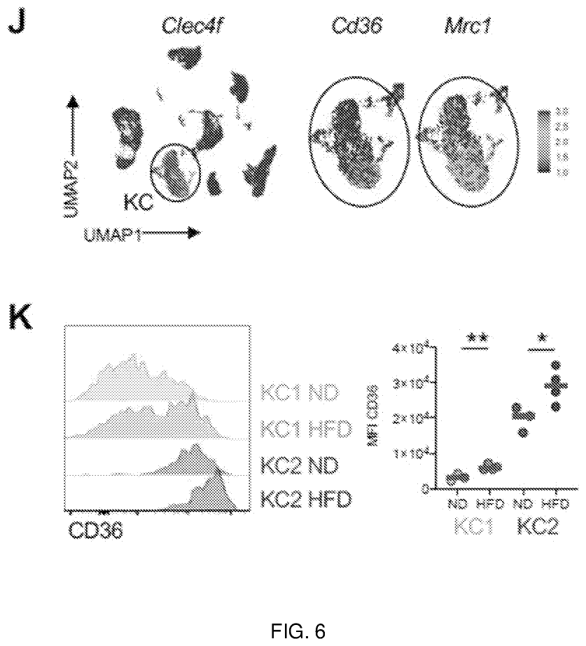

The inventors of the present disclosure further explored the significance of this pan-omics pronounced metabolic signature highlighted in the KC2 population. As the inventors of the present disclosure observed an upregulation of genes involved in carbohydrate and lipid metabolisms in KC2, the inventors of the present disclosure chose to use a model of high fat diet (HFD) feeding which induces obesity and its related metabolic disorders, including glucose intolerance and liver steatosis in mice (FIG. 6A). The inventors of the present disclosure noticed an increase in the relative frequency of the KC2 population as compared to mice fed with a normal diet (FIG. 6B).

As reported previously in other studies known in the art, the inventors of the present disclosure did not notice any significant monocyte recruitment into the livers of the monocyte fate-mapper Ms4a3^{cre}xRosa^{Tomato} mice in the 9 first weeks of HFD (FIG. 6C). The labeling of KC2 in the liver of Ms4a3^{cre}xRosa^{Tomato} mice under HFD increased after 18 weeks, suggesting that the growth of the KC2 population was partially dependent of monocyte recruitment only at late time point of HFD (FIG. 6C & 6D).

The inventors of the present disclosure then compared the transcriptomic profiles of the two populations of KCs sorted from mice fed with HFD for 2 months. The dichotomy between the two KC subpopulations was conserved (FIG. 6E) even if they noticed that both of them acquired a more pronounced metabolic-oriented signature upon HFD (FIG. 6F-I). Notably, genes involved in fatty acid processing were upregulated and the ones related to amino acid catabolismwere downregulated in KC2. The inventors of the present disclosure found Cd36 among the genes upregulated upon HFD, a gene extensively described in the literature for its role in lipid uptake and oxidative stress modulation in macrophages.

Accordingly, the inventors of the present disclosure checked the expression of Cd36 by KC2 in a recently and independently published murine liver single cell RNA-seq dataset. By focusing on the Clec4f+ KC population, the inventors of the present disclosure observed that Cd36 was more expressed in Mrc1+ KC (FIG. 6J). The inventors of the present disclosure then used conventional flow cytometry to validate these transcriptomic results and observed that the CD36 marker signal was indeed strongly elevated in KC2 compared to KC1 at steady-state and was upregulated upon HFD (FIG. 6K). These data highlighted KC2 as a metabolically responsive macrophage population with lipid handling being modulated during diet induced obesity.

### CD36-Specific Targeting in KC Modulates Liver Metabolism

To further study the role of KC2 in liver metabolism, the inventors of the present disclosure adopted an interventional approach by targeting KC2 in the context of metabolic challenges induced by HFD. As all macrophage-oriented fate-mapping models failed to discriminate KC1 and KC2, the inventors of the present disclosure did not specifically target KC2 with these tools. Therefore, the inventors of the present disclosure used the pan-omics signatures (FIG. 5) to identify KC2-specific markers that could be used to target them and to investigate their functional significance in HFD-fed mice.

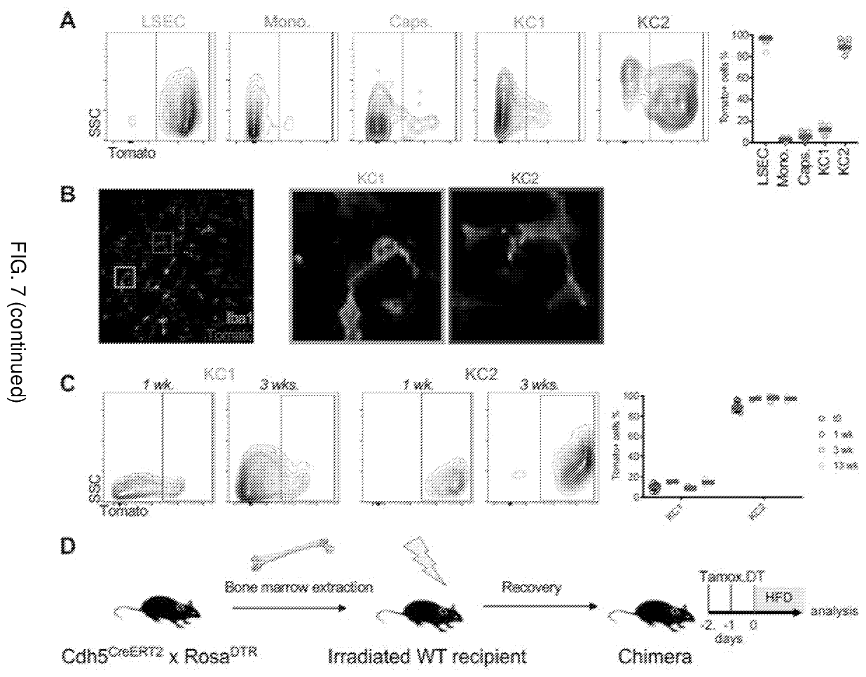

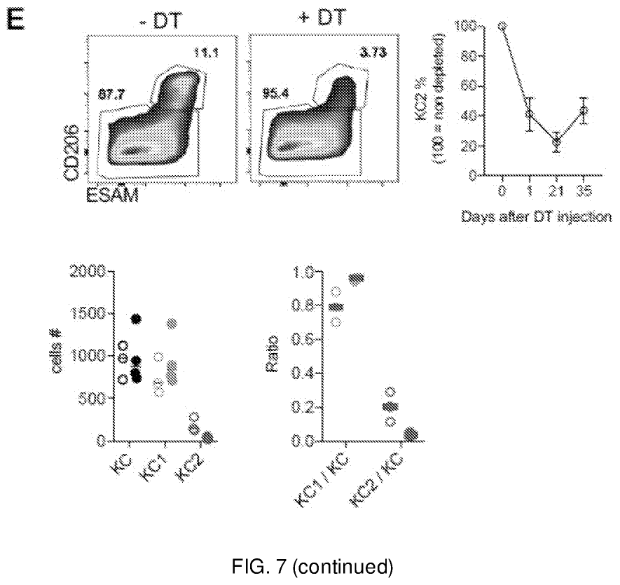

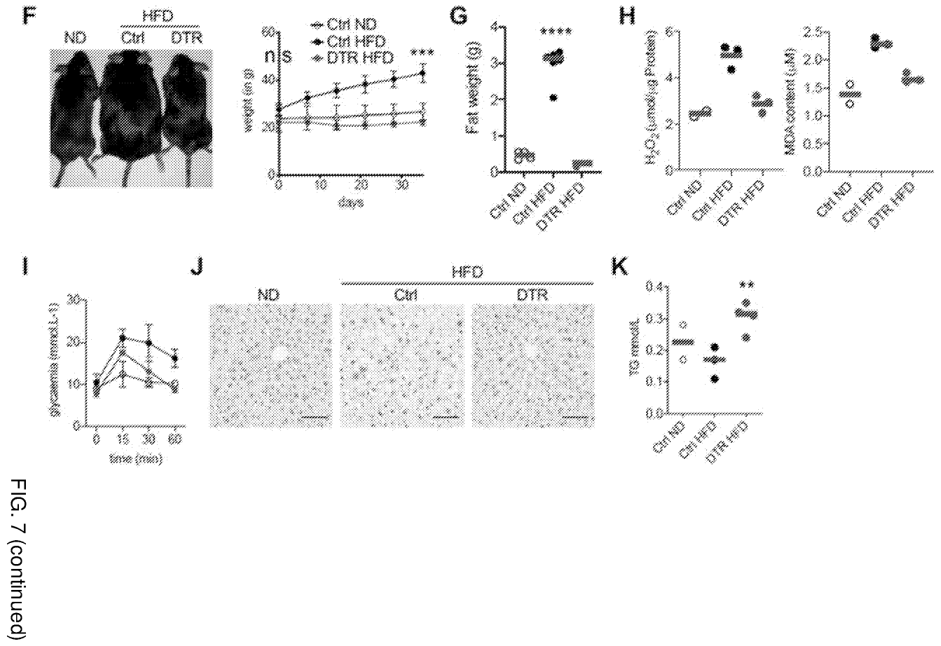

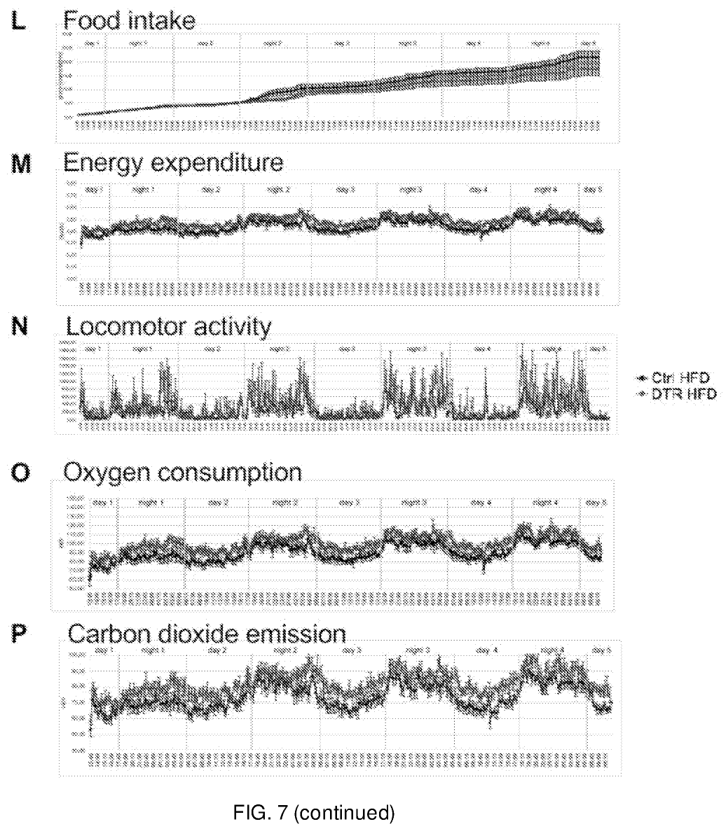

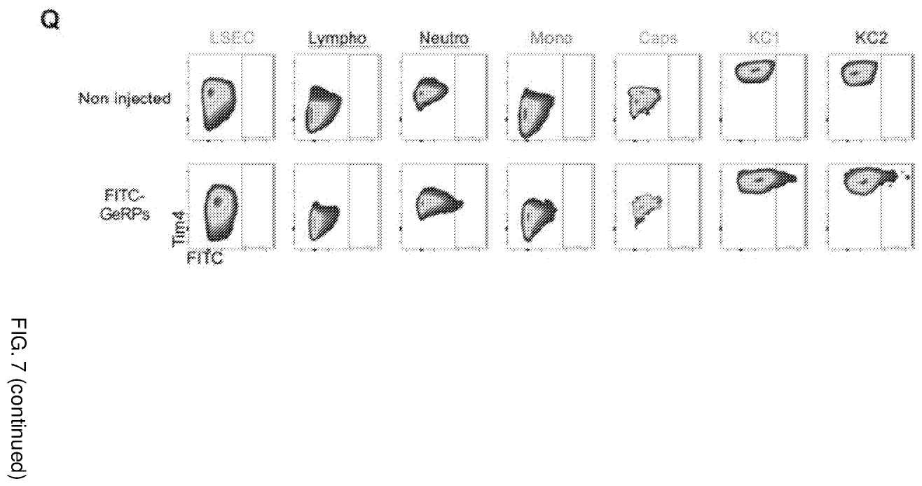

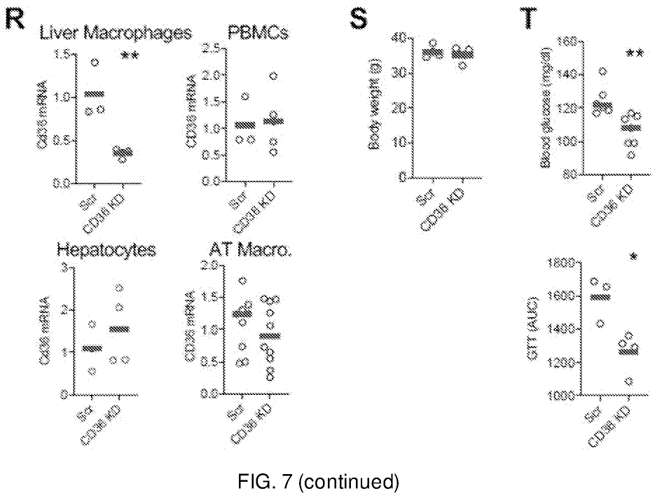

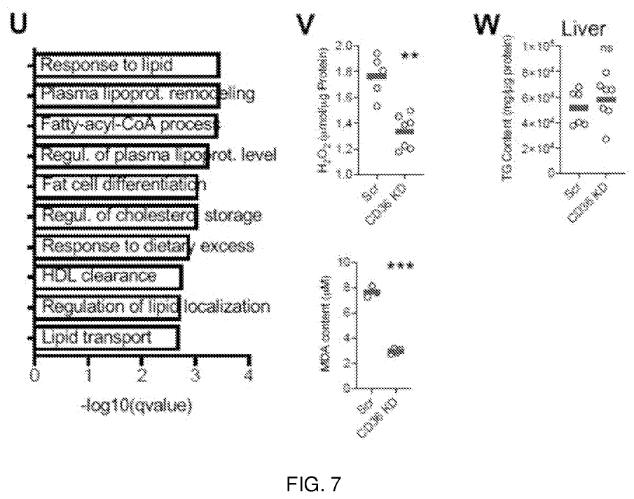

Cdh5 is considered as a core KC gene, but in parallel the Cdh5cre^{ERT2}xRosa^{Tomato} model has been successfully used to label endothelial cells. Cdh5 was more expressed in KC2 both at RNA and protein levels, and KC2, but not KC1, were specifically tagged in Cdh5^{creERT2}xRosa^{Tomato} mice fed with tamoxifen for 7 days (FIG. 7A & 7B). Of note, the labeling of the two subsets was very stable across time, with no observed changes for 13 weeks post-tamoxifen treatment, excluding a conversion from one subset to another one at steady state (FIG. 7C). Therefore, the inventors of the present disclosure made Cdh5^{creERT2}xRosa^{DTR} mice and generated chimeras (Cdh5cre^{ERT2}xRosa^{DTR} bone marrow engrafted into WT recipients to avoid targeting of radioresistant LSEC that also express Cdh5) to establish a system allowing inducible and specific depletion of KC2 (FIG. 7D). After tamoxifen induction, the inventors of the present disclosure validated this system by injecting Diphteria toxin (DT) into tamoxifen treated chimeric Cdh5cre^{ERT2}xRosa^{DTR} mice and followed the kinetics of KC2 depletion. Tim4+ CD206hi ESAM+ KC2 but not Tim4+ CD206lo ESAM− KC1 were efficiently depleted after one single injection of DT (FIG. 7E), while adipose tissue macrophages were not impacted for example supporting the specificity of such strategy to target KC2.

The inventors of the present disclosure then fed tamoxifen-and DT-treated Cdh5^{creERT2}xRosa^{DTR} chimeras (KC2-deficient group) with HFD for weeks to assess the role of KC2 in the HFD-driven metabolic changes. HFD did not induce any weight gain in KC2-deficient animals while KC2-sufficient animals gained weight over the period of HFD (FIG. 7F & 7G). Considering the noticeable regulation of genes involved in lipid metabolism and oxidative stress in KC2 upon HFD feeding, the inventors of the present disclosure monitored the amounts of both reactive oxygen species (ROS) H₂O₂, which is a well-known marker of oxidative stress, and the lipid peroxidation by-product Malondialdehyde (MDA) following KC2 depletion. As expected, HFD induced an increase of ROS and MDA (FIG. 7H). However, KC2 depletion resulted in a decrease of both ROS and MDA, suggesting that KC2 could contribute to hepatic lipid peroxidation and oxidative stress associated with obesity. This was accompanied with improved glucose tolerance and a less pronounced steatosis in KC2-depleted animals (FIGS. 7I & 7J). However, even if metabolic impairments including obesity, oxidative stress, steatosis and glucose intolerance were reduced, serum concentration of triglycerides (TG) was increased (FIG. 7K).

In line with these results, indirect calorimetric measurements revealed that KC2-depleted animals had lower food intake, higher energy expenditure related to respiratory exchange ratio of VO2 and VCO2 and an increased locomotor activity than KC2-sufficient during the early phase of HFD (FIG. 7L-P). Considering the entanglement of metabolic functions between different organs including notably the liver, pancreas, adipose tissue, and brain, these observations could reflect a profound metabolic rewiring consecutive to KC2 depletion and would deserve future detailed investigations.

However, to circumvent such possible systemic effects, the inventors of the present disclosure used a more targeted strategy based on Glucan-encapsulated siRNA Particles (GeRPs) for specific silencing of Cd36 in KCs. While the inventors of the present disclosure had previously shown that GeRPs are specifically delivered to macrophages in the liver, but not to macrophages or other cells in other organs, the inventors of the present disclosure first used FITC-GeRPs to verify the specificity of the targeting within the hepatic cell populations. When intravenously injected, FITC-GeRPs were only retrieved in KC1 and KC2 and not in other liver cells such as endothelial cells and monocytes confirming the equal phagocytic abilities of both KC subpopulations (FIG. 7Q). Then, mice were fed a HFD for 7 weeks and treated with GeRPs loaded either with a control scrambled siRNA or a Cd36 targeting siRNA for 2 weeks. This treatment allowed for the short-term specific silencing of Cd36 in KCs (FIG. 7R). Even if the inventors of the present disclosure did not observe any significant effect on weight gain during the 2-week treatment period (FIG. 7S), both glycaemia and glucose tolerance were improved in Cd36 siRNA treated animals (FIG. 7T).

To further investigate the effect of Cd36 on KC phenotype, the inventors of the present disclosure then analyzed the transcriptomic profile following Cd36 silencing and observed a modulation of genes involved in lipid metabolism (FIG. 7U). Finally, the inventors of the present disclosure tested the effect of Cd36 silencing on hepatic ROS and MDA concentrations. Consistent with the inventor of the present disclosure's hypothesis, Cd36 silencing decreased accumulation of both MDA and ROS (FIG. 7V).

This occurred independently of liver triglyceride content (FIG. 7W), Taken together, these data highlight a pivotal role of CD36 specifically expressed by KC2 in obese mice for liver metabolic homeostasis regulation.

Herein, the inventors of the present disclosure report the presence of two phenotypically and functionally distinct subsets of KCs in the healthy murine liver. The liver is now recognized as a common niche for several macrophage populations, but these two populations of KCs share a common embryonic origin clearly differentiating them from the other monocyte-derived liver macrophages. KC1 and KC2 are present at steady-state and are not the result of the recruitment of monocytes which occurs neonatally or in inflammatory. These two populations share the common core macrophage signature with a high expression of well-known KC-core markers including Clec4f, Lyz2, or Csf1r, while KC2 in addition express a set of genes previously thought to be restricted to LSEC.

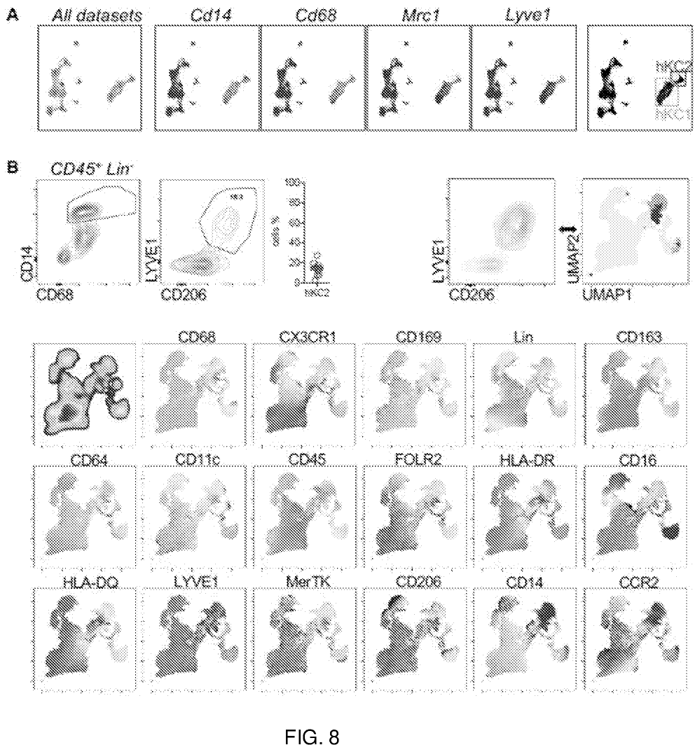

These results are in line with a previous study known in the art showing that cultured primary KCs share several functional antigens with LSEC; but the global approach used in the mentioned study did not allow the detection of a specific subpopulation of KCs. Although recent work by a study known in the art has detected potential contamination of KC populations by LSEC using conventional flow cytometry, the inventors of the present disclosure were able to identify and exclude this minor contamination in the single cell RNA-seq dataset. Thus, the KC2 population that the inventors of the present disclosure identified is composed of bona fide KCs, evidenced by fate-mapping models and the high expression of macrophage-core genes. In line with the current study of the present disclosure, the existence of a CD206+ population of KCs has been already reported in humans in a recently published study known in the art and validated by preliminary data generated by the inventors (FIG. 8). Furthermore, a Lyve1+CD206+ population of macrophages harbouring similarities with KC2 is also present in atherosclerotic plaques suggesting that such “metabolic” macrophages could be present in other niches than the liver. Of note, a role in active phagocytosis of blood-borne cellular material has been proposed by a study known in the art for the CD206+ macrophages. Together these data by studies known in the art argue for a central role of this receptor in macrophage biology and CD206 should be systematically included in future analyses of these cells.

This also parallels the recent findings of two populations of lung interstitial macrophages and across tissues discriminated by differential Lyve1 and CD206 expression. The other important biomarker that the inventors of the present disclosure have here identified is ESAM. This molecule, whose expression was thought to be restricted to LSEC, is also expressed by a subset of splenic DCs, but until now was not thought to be expressed by macrophages. Although Lyve1, Cd206 and Esam have been long considered as endothelial-restricted genes, the results of the present disclosure in the liver and previous ones in other organs are revisiting this concept, and clearly show that these genes can also be expressed by RTMs, even if the complete functional roles of these markers in RTMs need to be further investigated.

The dichotomy between CD206− and CD206+ populations is completely independent of the origin of the macrophages i.e monocyte- or embryonically-derived and should be dictated only by the niche of residence, reinforcing the concept of the niche being one dominant factor driving macrophage identity. However, here, the inventors of the present disclosure have failed to identify a distinct sub-liver niche for each subset. Metabolic zonation in the liver is important and shapes the transcriptomic pattern of hepatocytes and LSEC, but KC1 and KC2 seem to be randomly distributed within the acini, and thus their polarization is likely linked to unknown factors other than oxygen or nutrient availability. Identification of these determinants should extend the comprehension of what the inventors of the present disclosure can call the distinct “niches” present within the liver. This also stresses the need to investigate the heterogeneity of the other different cell types of the liver, including stellate cells and LSEC that could contribute to KC1 vs KC2 identity. Indeed, recent studies have shown in a mouse model of depletion of embryonic KCs, that liver-entering monocytes were reprogrammed into KC-like cells via crosstalk with hepatic stellate and LSEC. These two studies have identified a key liver triptych composed of hepatic stellate cells, LSEC and macrophages. Therefore, deciphering the crosstalk between these three different cell types and their potential subpopulations and notably how its modulation in the context of different liver pathologies will be a challenge for further studies.

In the context of the KC subsets identified here, the inventors of the present disclosure want to emphasize that KC2 are already present in the steady-state and are poised to respond to metabolic challenges. Therefore, determining how this cell identity is generated will be useful for understanding liver pathologies, considering that almost a quarter of the human population is affected by non-alcoholic fatty liver diseases. In line with this, the cellular liver triptych also seems to be present in humans as evidenced by the identification of key “stellakines”, which are secreted by hepatic stellate cells during NASH and cirrhosis and impact macrophage biology. As exemplified in studies known in the art, the recent progress of single cell transcriptomics and the subsequent release of several atlases in both mouse and human will improve our knowledge of hepatology and liver diseases, even if the existence of corresponding functionally distinct KC1 and KC2 populations in humans remains to be formally established.

Functionally, the inventors of the present disclosure identified a specific metabolic role for KC2. However, it would be misleading to claim that only KC2 are involved in liver metabolism regulation. The data of the present disclosure rather highlight that both KC subpopulations participate to this function, even if KC2 population seems to be more wired to do it. Furthermore, it is noticeable that KC2 is present in steady state and do not depend of the monocyte recruitment that occurs in the late stages of obesity. In line with this, a previous study known in the art indicated a central role of liver macrophages in metabolism considering KCs as a homogeneous population. The authors of the study known in the art showed that the non-inflammatory factor insulin-like growth factor-binding protein 7 (IGFBP7) was important in metabolic regulation by KCs. Importantly, in addition to Cd36, the inventors of the present disclosure observed that Igfbp7 was among the top differentially expressed genes between KC1 and KC2, being highly over-expressed in KC2. Hence, it is tempting to hypothesize that the effect reported in the study could be mostly driven by the KC2 population. In addition, the inventors of the present disclosure provide direct evidence for a role of the KC2-expressed receptor CD36 in liver metabolism regulation. These results are perfectly in line with the emerging central role of CD36 in NASH development and obesity-related disorders. While further investigations are needed to better understand the systemic effect of KC2 depletion, our targeted approach to silence the lipid transporter CD36 revealed an important role of these cells in the regulation of glucose homeostasis and oxidative stress. It has been previously reported that decreasing oxidative stress in KCs in obesity can improve liver metabolism and decrease ROS concentrations. Here the inventors of the present disclosure refine this observation by showing that KC2 play a major role in this process by processing lipids via CD36.

However, it should recognise that a precise understanding of NASH aetiology is not yet achieved and that humans probably develop a spectrum of convergent diseases. So, it cannot be excluded that KCs could be differentially involved in other experimental models of NASH. For example, it has been shown that inflammatory monocytes enter into the liver and induces transient changes in liver macrophage homeostasis in a NASH model induced by a methionine-choline deficient (MCD) diet. Furthermore, very recent studies known in the art using different diets report that embryonically derived KCs disappeared after several weeks and are replaced by a population of monocyte-derived lipid-associated macrophages (LAM) linked with the development of pathologies and comparable to the ones already reported in adipose tissue from obese individuals. However, these studies used essentially rodent-based models which by definition can be limited in their abilities to mimic human diseases. As an example, MCD-induced NASH is associated to a severe and quick loss of weight, liver inflammation and subsequent fibrosis. Even if the latter could be close to the human situation, the first is not observed clinically and might be responsible of model-specific side-effects. On the contrary, HFD-induced NASH could reflect more human obesity-induced disorders but is less efficient to induce liver fibrosis. Therefore, it appears fundamental to remind that there is not yet a model universally recognised to fully recapitulate human NAFLD/NASH. This being said, the model of HFD that the inventors of the present disclosure used herein does not induce a significant recruitment of monocytes in the liver at early stages. So, while most of the studies were focused on monocyte-derived LAM recruited in the metabolically challenged liver, the study of the present disclosure indicates that a fraction of embryonic KCs is already fated to assume metabolic functions. Further studies should refine their precise roles in this context or even in other ones, allowing for the design of innovative therapies targeting metabolic function of KC2 for the modulation of liver metabolic diseases.

Finally, in parallel to the current study in which the inventors of the present disclosure described the two distinct KC1 and KC2 populations and KC2 metabolic functions, the inventors of the present disclosure have investigated their capacities to present antigens in a mouse model of hepatitis B virus pathogenesis. This has revealed key differences between the two populations with KC2 being notably responsive to IL-2 signalling and involved in mounting efficient T-cell mediated responses to hepatocellular antigens. This study reports a specific effect of the KC2 subpopulation in mounting immune responses and validates our approach of investigating heterogeneity of tissue resident macrophage populations. Indeed, even if the literature often assumes that macrophages from one common tissue compose a uniform population, the data reported in the present disclosure and previous studies known in the art have shown that subtissular niches exist, inhabited by different macrophage populations. Thus, development of macrophage-based therapeutic strategies will have to take this heterogeneity into account to improve the specificity and efficiency of innovative treatments.

In summary, the inventors of the present disclosure aimed at clarifying KC heterogeneity by combining single cell transcriptomics to specific monocyte fate-mapping models and functional validation and used high-dimensional approaches to characterize macrophage populations in the murine liver. The inventors of the present disclosure identified two distinct populations among embryonically-derived Kupffer cells (KC) in the steady-state murine liver sharing a core signature while differentially expressing numerous genes and proteins: a major CD206loESAM− population (KC1) and a minor CD206hiESAM+ population (KC2). The inventors of the present disclosure confirmed the common embryonic origin of these populations and their independence from inflammatory monocytes and Tim4− capsular macrophages.

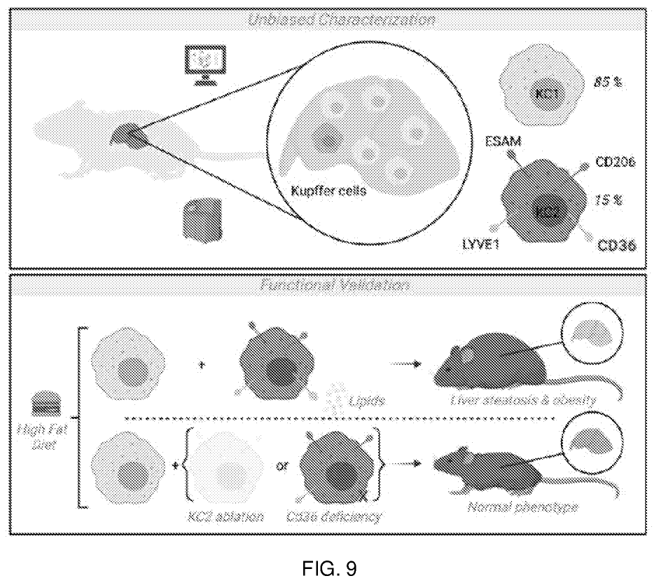

Functionally, the inventors of the present disclosure found that KC1 and KC2 have specific transcriptomic and proteomic signatures. KC2 expressed genes involved in metabolic processes including fatty acid metabolism in both steady state and in diet-induced obesity and hepatic steatosis. CD206hi ESAM+ KC2 are involved in the regulation of liver metabolism in a murine model of obesity via their high expression of the fatty acid transporter CD36. Functional characterization by depletion of KC2 or targeted silencing of the fatty acid transporter Cd36 highlighted a crucial contribution of KC2 in the liver oxidative stress associated with obesity (FIG. 9). This study reveals that KCs are more heterogeneous than anticipated notably describing a subpopulation wired with metabolic functions.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

FIG. 1A shows plots and heatmaps representing gene expressions. CD45+ Tomato-Liver cells were extracted from healthy Ms4a3^{cre}xRosa^{Tomato} mice and libraries of mRNA were generated and sequenced by using the Chromium technology. Seurat analysis was conducted defining 9 clusters with distinct patterns of gene expression. Each dot corresponds to a single cell, colored according to the clusters identified. Expression of few representative genes is overlaid to define each cluster and a heatmap of the most highly differentially expressed genes (DEGs) in the different clusters is displayed. The following genes are among the most highly expressed in the respective clusters: Cd79a and Igkc in cluster 0, Cd3g and Trac in cluster 1, Id3 and Mrc1 in cluster 2, C1qb and Lyz2 in cluster 3, Cd9 and Dpp4 in cluster 4, Nkg7 and Ccl5 in cluster 5, Ctla4 and Gzmk in cluster 6, Siglech and Runx2 in cluster 7, and Cx3cr1 and Cd14 in cluster 8.

FIG. 1B shows plots with a zoom on the Adgre1+ macrophage population showing the Cx3cr1+ capsular macrophages (Caps.) and the two clusters of Timd4+ Clec4f+ KCs (KC-c1 & KC-c2). Violin plots in this figure show expression of selected genes in the two KC clusters.

FIG. 1C shows a plot of tSNE projection of sorted CD45+ CD64+ F4/80+ liver cells sequenced according to SMARTseq2 protocol. Each dot corresponds to a single cell, colored according to the clusters identified. KC clusters correspond to c3 and c4.

FIG. 1D shows plots of tSNE projection of sorted CD45+ CD64+ F4/80+ liver cells sequenced according to SMARTseq2 protocol. The expression of indicated macrophage-specific genes is overlaid.

FIG. 1E shows a dot plot of the integration of SMARTseq2 (probes) and Chromium (reference) datasets focused on Clec4f+ KCs for validation of the clustering.

FIG. 1F show plots of scenic analysis of the high-resolution SMARTseq2 dataset with the overlay of the 4 clusters identified in the Seurat analysis. Two stable states within the macrophage population corresponding to Seurat c3 and c4 are visible. Number of genes included in each regulon is provided in parentheses.

FIG. 1G shows plots of scenic analysis of the single cell RNA-seq SMARTseq2 dataset, with violin plots of representative regulons from each Seurat-defined cluster.

FIG. 1H shows a dot plot representation of the expression of the indicated markers projected onto a tSNE analysis showing the different clusters in live liver CD45+ singlets analysed with a 37-marker extended CyTOF panel. The unsupervised analysis was done with the Phenograph algorithm and revealed 15 clusters, manually assigned as indicated populations thanks to lineage markers. Dot plot representations of the level of expression of the indicated markers projected on a tSNE analysis. Different clusters of liver alive CD45+ singlets analysed by a 37-markers CyTOF panel are shown (13 most representative markers are depicted).

FIG. 1I shows dot plot representations of the level of expression of the indicated markers projected on a tSNE analysis. Different clusters of liver alive CD45+ singlets analysed by a 37-markers CyTOF panel are shown (the 24 additional markers are depicted).

FIG. 1J shows plots and heatmaps of the CyTOF analysis with the OneSENSE algorithm of live liver CD45+ singlets. F4/80+ Tim4+ KC cells are shown within a dashed black frame.

FIG. 2A shows flow cytometry plots with gating strategy that is used to analyse liver cells. LSEC are defined as CD45low CD31+, macrophages as CD45+ Lin− F4/80+ CD64+, monocytes as CD45+ Lin− F4/80− CD64hi Ly6Chi, caps. macs as CD45+ Lin− F4/80+ CD64+ Tim4− MHCIIhi and KCs as CD45+ Lin−F4/80+ CD64+ Tim4hi MHCIIint cells.

FIG. 2B shows flow cytometry analysis, with the sorting strategy for bulk RNA sequencing and volcano plots on the 200 most expressed genes among differentially expressed genes (p value <0.001), between CD206lo CD107b-KC1 and CD206hi CD107b+ KC2 from bulk RNA sequencing analysis. Each dot denotes a differentially expressed gene. The ratio between nucleus and cytoplasm areas from sorted cells is shown.

FIG. 2C shows flow cytometry plots of Tim4hi KCs and MHCIIhi capsular macrophages among total macrophages (left) and CD206lo ESAM− KC1 and CD206hi ESAM+ KC2 among KCs (right). For the quantification, each dot represents an individual and the median is indicated by a line.

FIG. 2D shows flow cytometry plots of MHCIIhi capsular macrophages, CD206lo ESAM− KC1 and CD206hi ESAM+ KC2. The expression of each indicated marker is displayed.

FIG. 2E shows dot plot representation of the level of expression of the indicated markers projected on a tSNE analysis of the live liver singlets. Data were analysed by a 11-marker flow cytometry panel showing manually-defined KC2 (red), KC1 (blue) and LSEC (green).

FIG. 2F shows scanning electron and optical (cytospin) microscopy images of flow cytometry sorted liver KC1, KC2, capsular macrophages and monocytes. Scale bars represent 1 μm.

FIG. 2G shows plots with the kinetics of the relative abundance of the different liver cell populations upon development, from birth (Od) to 8 week-old.

FIG. 2H shows flow cytometric measurement of the frequency of Tomato expression in indicated populations in 8-week-old Ms4a3^{cre}xRosa^{Tomato} mice. Each dot represents an individual and the median is indicated by a line.

FIG. 2I shows representative flow cytometry plots from the analysis of S100a4^{Cre}xRosaE^{YFP} and Csf1r^{GFP} mice. Each dot represents an individual and the median is indicated by a line.

FIG. 2J shows representative flow cytometry plots from the analysis of the CD45.2 parabiont from a couple of CD45.1/CD45.2 parabiotic mice. WT CD45.1 were surgically attached to WT CD45.2 and analysed after 3 months. Each dot represents an individual and the median is indicated by a line.

FIG. 3A shows representative flow cytometry plots from the analysis of livers of C57BL/6 WT mice intravenously injected with anti CD45 (500 ng per mouse) 5 min before sacrifice. Each dot represents an individual and the median is indicated by a line. Non-injected controls and injected ones are shown.

FIG. 3B shows low magnification immunofluorescence microscopy images of liver vibratome sections from WT C57BL/6 mice. Sections are labelled for Clec4F and CD206 and stained with DAPI. Scale bars represent 20 μm.

FIG. 3C shows high magnification Immunofluorescence microscopy images of liver vibratome sections from WT C57BL/6 mice. Sections are labelled for Clec4F and CD206 and stained with DAPI. Scale bars represent 10 μm.

FIG. 3D shows single z-slices of fluorescent micrographs of single channels (CD206—red and F4/80—green) and merged images including DAPI-blue) depicting examples of KC1 and KC2 after in vivo CD206 labelling. Scale bars represent 5 μm.

FIG. 3E shows a scheme of the generation of the CD206^{Cre}xRosa^{Tomato} mice.

FIG. 3F shows immunofluorescence microscopy images of liver sections from Mrc1cre^{ERT2}xRosa^{Tomato} mice treated with a single injection of tamoxifen 24 h before analysis. Sections are labelled for F4/80 and stained with DAPI. Scale bars represent 20 μm. 32 Quantification of F4/80+CD206lo (KC1) and F4/80+CD206hi (KC2) cells on independent fields is displayed.

FIG. 3G shows images of liver section labelled for F4/80, CD206 and Cytokeratin 7. The distance between KC1 or KC2 and portal triads was measured and plotted.

FIG. 4A shows flow cytometry plots of the total liver onto which CD45− CD31+ LSEC, CD45+ Lin− F4/80+ CD64+ Tim4+ ESAM−CD206lo KC1 and CD45+ Lin−F4/80+ CD64+ Tim4+ ESAM+CD206hi KC2 are projected.

FIG. 4B shows flow cytometry profiles of the intensity of expression of indicated markers on the three populations.

FIG. 4C shows imaging cytometry (Image stream) analysis of liver cells. KC1 (blue-top) and KC2 (red-bottom) are shown.

FIG. 4D shows scanning electron microscopy images of sorted liver LSEC and KC2. Scale bars represent 1 μm. Fenestrae indicated by white arrows are shown in the magnified image.

FIG. 4E shows flow cytometric measurement of the frequency of YFP expression in indicated populations in 8-week-old Lyz2crexRosaYFP mice. KC1 (bottom square) and KC2 (top square) are overlaid at each step of the gating strategy. Each dot represents an individual and the median is indicated by a red line.

FIG. 4F shows plots of immune cell populations manually annotated based on the expression of the indicated markers. CD45+ liver cells, KC1 and KC2 were sorted and loaded on a BD Rhapsody cartridge following manufacturer recommendations. The immune response panel Mm (BD) was used allowing the monitoring of expression of 397 genes and the generation of the tSNE.

FIG. 4G shows flow cytometric measurement of the frequency of LSEC, KC1 and KC2 populations in C57BL/6 mice after clodronate liposome (CLL) mediated KC depletion. Each dot represents an individual and the median is indicated by a line.

FIG. 4H shows flow cytometric measurement of the frequency of KC1 and KC2 populations in Ms4a3^{cre}xRosa^{Tomato} mice after clodronate liposome-mediated KC depletion. Each dot represents an individual and the median is indicated by a line.

FIG. 5A shows plots of principal component analysis of the transcriptomes from bulk RNA sequencing of sorted liver KC1 and KC2.

FIG. 5B shows a dot plot representation of the expression of genes expressed by KC1 and KC2. Genes known to be highly expressed in macrophages are Clec4f, Lyz2, Csf1r, Timd4, and ones described to be predominantly expressed in LSEC are Mrc1, Pecam1, Esam and Cdh5.

FIG. 5C shows a heatmap of the DEGs between the two populations. For the heatmap, KC2 have higher expression of 1364 genes and lower expression of 51 genes compared to KC1 population.

FIG. 5D shows heatmap of selected genes representative of macrophages or endothelial cells expressed in sorted KC1, KC2 or LSEC.

FIG. 5E shows principal component analysis of the bulk RNAseq data generated after sorting of LSEC, KC1 and KC2.

FIG. 5F shows Venn diagram of the 100 most expressed genes in KC1 and KC2. There are 64 common genes among the 100 most expressed genes including the indicated canonical macrophage genes.

FIG. 5G shows volcano plots of the differentially expressed genes between sorted LSEC and KC1, or LSEC and KC2, highlighting conserved endothelial vs. KC (both subsets) signatures.

FIG. 5H to 5J shows plots and heatmaps with the same analysis as in FIG. 5A to FIG. 5C, but with the translatomes obtained from Lyz2^{cre}×Rp/22^{HA} mice (RiboTag approach). For the heatmap, KC2 have higher expression of 309 genes and lower expression of 98 genes compared to KC1 population.

FIG. 5K to 5M shows plots and heatmaps with the same analysis as in FIG. 5A to FIG. 5C, but with proteomes. For the heatmap, KC2 have higher expression of 509 proteins and lower expression of 32 proteins compared to KC1 population.

FIG. 5N shows plots with principal component analysis of the integrated transcriptome, translatome and proteome datasets.

FIG. 5O shows Venn diagrams of the 100 most expressed genes/proteins identified from the different technologies.

FIG. 5P shows RNA-seq based alluvial plots of KC1- or KC2-specific general (left) or metabolism-related (right) pathways.

FIG. 5Q shows RNA-seq based integrated network analysis of KC2 as compared to KC1 at steady-state. Network-based integration of gene expression datasets was conducted as described by other studies known in the art. Briefly, topological tool for Integrated Network Analysis was mapped into KEGG pathway. Up and down regulated metabolic genes based on false discovery rate (FDR) were mapped into models maintaining all essential KEGG pathway attributes.

FIG. 5R shows heatmaps of the top 10 metabolism related DEGs between KC1 and KC2.

FIG. 6A shows images of hematoxylin and eosin staining of normal diet (ND) or high-fat diet (HFD) fed mouse livers after nine weeks of diet.

FIG. 6B shows flow cytometry plots of the frequency of KC1 and KC2 among KCs in mice fed with HFD for the indicated time. Each dot represents an individual and the median is indicated by a line.

FIG. 6C shows representative flow cytometry plots from the analysis of Ms4a3^{Cre}xRosa^{Tomato} mice upon HFD for the indicated timepoints. Each dot represents an individual and the median is indicated by a line.

FIG. 6D shows similar flow cytometry plots seen in FIG. 6C, but with gating on KC1 and KC2 populations.

FIG. 6E shows a heatmap of the top DEGs between KC1 and KC2 across different diets (ND Normal Diet, HFD High-Fat Diet, MCD Methionine-Choline Deficient Diet).

FIG. 6F shows RNA-seq based analysis of KC1 and KC2 focused on uptake of LDL and lipid storage.

FIG. 6G shows pathway analysis using total DEGs between KC1 or KC2 sorted from ND and HFD-fed mice.

FIG. 6H shows heatmap and pathway analysis of the DEGs between KC1 sorted from ND or HFD mice. Canonical DEG are displayed in the boxes.

FIG. 6I shows heatmap and pathway analysis of the DEGs between KC2 sorted from ND or HFD mice. Canonical DEG are displayed in the boxes and integrated network analysis of DEGs between ND and HFD is provided.

FIG. 6J shows plots of single cell RNAseq data (55, 118 cells) extracted from a study known in the art. KCs were identified due to their high expression of Clec4f. Cd36 and Mrc1 expression are overlaid on the KC population.

FIG. 6K shows flow cytometry profiles of the intensity of expression of CD36 on the indicated populations and quantification of the MFI.

FIG. 7A shows representative flow cytometry plots from the analysis of Cdh5^{CreERT2}xRosa^{Tomato} mice after 1 week of tamoxifen-enriched diet for induction of the recombination. The percentage of positive cells is displayed for the indicated populations of liver cells.

FIG. 7B shows the imaging of chimeric Cdh5^{CreERT2}xRosa^{Tomato} mice. The liver was processed and sectioned with a vibratome (300 μm thick slices) and stained for Iba-1 and Tomato.

FIG. 7C shows representative flow cytometry plots from the analysis of Cdh5^{CreERT2}xRosa^{Tomato} mice after the indicated timepoints from the end of the tamoxifen-diet induction.

FIG. 7D shows a schematic representation of the generation of KC2-depleted mice.

FIG. 7E shows flow cytometric analysis of liver KCs in Cdh5^{CreERT2}xRosa^{Tomato} mice. Specific ablation of KC2 depletion was monitored at the indicated timepoints after one injection of DT.

FIG. 7F shows an image of mice after 6 weeks of HFD. The absolute weight in control and KC2-depleted mice during the 6 first weeks of HFD.

FIG. 7G shows a plot with the white adipose tissue weight in control and KC2-depleted mice after 6 weeks of HFD.

FIG. 7H shows plots of hydrogen peroxide and malondialdehyde assays in the liver of the indicated mice.

FIG. 7I shows a plot with glucose tolerance test that was performed on overnight fasted mice during week 6 of HFD.

FIG. 7J shows images of hematoxylin and eosin staining of liver slices from indicated mice.

FIG. 7K shows a plot with measurements of circulating triglycerides in indicated mice.

FIG. 7L to 7P shows plots with energy intake and expenditure measurements of mice placed individually in metabolic cages during the first week of the HFD.

FIG. 7Q shows flow cytometry analysis plots. Mice were i.v injected with unloaded FITC-labeled glucan-encapsulated siRNA particles (GeRPs) and liver cells were analyzed 24 h later.

FIG. 7R shows plots of Cd36 expression that was assessed by qPCR at the end of the treatment on indicated sorted cells. Mice were injected with GeRPs containing scrambled RNA (Scr) or siRNA directed against Cd36 (si-CD36) thrice a week for 2 weeks.

FIG. 7S shows a plot of the body weight at the end of the GERPs treatment.

FIG. 7T shows plots of glycemia and glucose tolerance test (GTT) measured at the end of the treatment.

FIG. 7U shows pathway analysis comparing data from CD36 KD and Scr-treated mice, with pathways modulated displayed. Bulk RNA-seq was performed on total liver macrophages.

FIG. 7V shows plots with measurement of oxidative stress markers MDA and H2O2 in the liver of the same mice. Each dot represents an individual and the median is indicated by a line.

FIG. 7W shows a plot of the liver concentration of triglycerides from CD36 KD and Scr-treated mice.

FIG. 8A and FIG. 8B shows UMAP projection obtained by analyzing CD45⁺ cells (PTPRC-expressing) from several human liver datasets available in the literature. Unsupervised clustering was performed using the Seurat analysis pipeline. Feature plot representation of the normalized expression level of macrophage markers (Cd14 & Cd68) and KC2 markers (Mrc1 & Lyve1) on the UMAP with integrated samples.

FIG. 9 shows schematic diagrams with summaries of the current application.

### APPLICATIONS

Embodiments of the methods disclosed herein provide an accurate marker for detecting sub-populations of macrophages. Embodiments of the disclosed methods also seek to provide a method of determining the risk of obesity and/or a metabolic impairment related to obesity.

Advantageously, the present disclosure provides a method modulating obesity comprising an active depletion/targeting of a subspopulation of Kupffer cells exhibiting metabolic functions.

Even more advantageously, the present disclosure provides a Cdh5^{creERT2_}xRosa^{Tomato} mouse model in which the role of obesity-controlling liver macrophages can be easily assessed.

The present disclosure also provides the diagnostic use of Igfbp7/Cd36 expression levels in liver macrophages as a susceptibility marker of developing obesity.

The present disclosure also provides a therapeutic use of the method and/or mixture as disclosed herein through the development of specific drugs inhibiting Igfbp7/Cd36 expression or the related downstream pathway.

The present disclosure also provides unbiased high-throughput approaches that reveal two subsets of murine Kupffer cells (KC).

The present disclosure also provides a method of characterising a distinct Kupffer cell population having CD206hiESAM+ KC2 cells that exhibit a distinct metabolic signature.

The present disclosure also advantageously provides for a method of depleting metabolically-wired KC2 subset prevents diet-induced obesity

The present disclosure also provides an insight that CD36hi KC2 regulates liver oxidative stress associated with obesity via CD36 expression. Therefore, the present disclosure provides for methods of improving a subject suffering from liver oxidative stress associated with obesity.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of detecting a population of macrophage in a sample, comprising: detecting and/or determining the expression of Cdh5 in the macrophage in the sample.

2. The method of claim 1, wherein the method further comprises detecting and determining the expression of one or more markers comprising CD107a, CD107b, IGFBP7 (Insulin-like growth factor-binding protein 7), LYVE1, CD36, CD206 and/or ESAM in a macrophage in the sample.

3. The method of claim 1, wherein method further comprises detecting and determining a cell to be a macrophage in the sample by detecting the expression of a macrophage marker.

4. The method of claim 1, wherein the macrophage is a Kupffer cell (KC), optionally an embryonically derived Kupffer cell.

5. The method of claim 1, wherein the method further comprises detecting and determining a cell to be a macrophage in the sample by detecting the expression of Clec4f, Lyz2, Vsig4, Csf1r, Adgre1, F4/80, Tim4, Clec4F, and Vsig4.

6. The method of claim 1, wherein the method comprises detecting and determining a population expressing CD206lo and/or ESAM− to be a first population of a Kupffer cell and a population expressing CD206hi and/or ESAM+ to be a second population of a Kupffer cell.

7. The method of claim 1, wherein the method comprises detecting and determining a population expressing CD206lo and ESAM− to be a first population of a Kupffer cell and a population expressing CD206hi and ESAM+ to be a second population of a Kupffer cell.

8. The method of claim 1, wherein an over-expression of one or more markers comprising CD107a, CD107b, IGFBP7 (Insulin-like growth factor-binding protein 7), LYVE1, CD36, CD206 and/or ESAM determines a population to be a second population of a Kupffer cell.

9. The method of claim 1, wherein the method further comprises detecting, sorting, and/or determining the presence of one or more marker comprising CD206, ESAM, CD36, and combinations thereof.

10. The method of claim 1, wherein the method further comprises separating the first and/or the second population of macrophage, optionally wherein the method further comprises separating the first and/or the second population of a Kupffer cell.

11. The method of claim 1, wherein the method further comprises removing the population of cells expressing one or more of Cdh5+, CD107b+, CD206hi and/or ESAM+ from the sample.

12. The method of claim 1, wherein the method further comprises determining the expression of one or more markers comprising CD45, CD64, F4/80, TIM4, Clec4F, Adgre1 (F4/80), Timd4, Csf1r, and Clec4f, optionally the method further comprises removing and/or excluding cells that express one or more Adgre1+, Cx3cr1+, Timd4−, Clec4f−, and combination thereof.

13. A kit for detecting and/or separating and/or depleting a population of a macrophage, comprising:
providing an agent for detecting a population of a macrophage expressing Cdh5,
optionally providing an agent capable of separating the population of the macrophage expressing Cdh5, and
optionally providing an agent capable of depleting the population of the macrophage expressing Cdh5.

14. The kit of claim 13, wherein the kit further provides an agent for detecting a population of a macrophage expressing CD107b+, CD206hi and ESAM+,
optionally providing an agent capable of separating the population of the macrophage expressing CD107b+, CD206hi and ESAM+, and
optionally providing an agent capable of depleting the population of the macrophage expressing CD107b+, CD206hi and ESAM+.

15. A transgenic animal model, comprising:
a macrophage population expressing Cdh5 that have been genetically engineered to undergo ablation upon exogenous activation.

16. The method of claim 1, wherein the method further comprises depleting a population of a macrophage, wherein said depleting comprises:
detecting and reducing a population of the macrophage in the subject, wherein the population of the macrophage expresses one or more Cdh5, CD107b, CD206, and ESAM, optionally wherein the method reduces a CD206hi and ESAM+ macrophage, optionally the method reduces a Cdh5+, CD206hi, and ESAM+ Kupffer cell.

17. The method of claim 1, wherein the method further comprises improving the health of an obese and/or overweight subject, wherein the improving comprises:
reducing a population of a macrophage in the subject,
wherein the population of the macrophage expresses one or more Cdh5, CD107b, CD206, and ESAM,
optionally wherein the method reduces a CD206hi and ESAM+ macrophage, optionally the method reduces a Cdh5+, CD206hi, and ESAM+ Kupffer cell

18. The method of claim 1, wherein the method further comprises determining the risk of obesity and/or a metabolic impairment related to obesity in a subject, wherein the determining comprises:
detecting the expression level of Igfbp7/Cd36 expression in a macrophage.

19. The method of claim 18, wherein the method further comprises treating the subject identified to be of risk of obesity and/or the metabolic impairment related to obesity in the subject with an agent capable of depleting a macrophage cell expressing Cdh5.

20. The method of claim 16, wherein the method reduces a CD206hi and ESAM+ macrophage, optionally the method reduces a Cdh5+, CD206hi, and ESAM+ Kupffer cell.
